(19) United States
(12) Patent Application Publication
(10) Pub. No.: US 2025/0313537 A1
(43) Pub. Date: Oct. 9, 2025

(54) BENZODIAZEPINE ANALOGS AND METHODS OF USE IN TREATING CANCER

(71) Applicants: University of Cincinnati, Cincinnati, OH (US); UWM Research Foundation, Inc., Milwaukee, WI (US)

(72) Inventors: Soma Sengupta, Cincinnati, OH (US); Daniel Pomeranz Krummel, Cincinnati, OH (US); Donatien Kamdem Toukam, Cincinnati, OH (US); James Cook, Whitefish Bay, WI (US); Taukir Ahmed, Milwaukee, WI (US); Sepideh Rezvanian, Milwaukee, WI (US); Laura Kallay, Cincinnati, OH (US)

(73) Assignees: University of Cincinnati, Cincinnati, OH (US); UWM Research Foundation, Inc., Milwaukee, WI (US)

(21) Appl. No.: 18/865,006

(22) PCT Filed: May 12, 2023
(86) PCT No.: PCT/US2023/022083

§ 371 Date: Nov. 12, 2024

Related U.S. Application Data
(60) Provisional application No. 63/341,571, filed on May 13, 2022.

(51) Int. Cl. C07D 243/24; A61K 31/5513; A61K 31/5517; A61P 35/00; C07D 243/22; C07D 487/04

(52) U.S. Cl. CPC C07D 243/24; A61K 31/5513; A61K 31/5517; A61P 35/00; C07D 243/22; C07D 487/04

(57) ABSTRACT

Provided herein are benzodiazepine analogs that are modified at the 7-position on the benzodiazepine ring to include a moiety selected from C₂-C₄ alkynyl, C₃-C₆ cycloalkyl, d(5)-cyclopropyl, methyl alkynyl, alkynyl-CD₃, and alkynyl-CF₃. Also provided are methods of use of the compounds in treating cancer, sensitizing a tumor to radiation, sensitizing a tumor to immunotherapy or chemotherapy, and treating neurological conditions associated with Type-A GABA neurotransmitter receptor function. Pharmaceutical compositions including the compounds are also provided.

### CROSS REFERENCE TO RELATED APPLICATION

This application claims priority to U.S. Provisional Application Ser. No. 63/341,571, filed May 13, 2022, the entire contents of which are incorporated herein by reference.

### TECHNICAL FIELD

The present disclosure relates to the field of benzodiazepine analogs and their therapeutic use.

### BACKGROUND

GABA, or γ-aminobutyric acid, is an amino acid that functionally acts as a neurotransmitter and is critical to neurotransmission. Type-A GABA neurotransmitter receptors are a major inhibitory neurotransmitter receptor in the mammalian central nervous system (CNS), but these same receptors are also present outside of the CNS. Genes coding for subunits of Type-A GABA neurotransmitter receptors are expressed in disparate cancer cells and it has been shown that cancer cells possess intrinsic functional Type-A GABA neurotransmitter receptors.

Type-A GABA neurotransmitter receptors (GABA_{A} receptors) are significant pharmacologic targets for the treatment of various neurological disorders, including anxiety and epilepsy. Among the therapeutic agents that work through acting on the Type-A GABA neurotransmitter receptors are the benzodiazepines, which bind at the interface between the alpha and gamma subunits of the pentameric structure (see FIG. 1). Benzodiazepines function to enhance the effectiveness (i.e., chloride anion transport) of GABA, the natural ligand of Type-A GABA neurotransmitter receptors.

A need exists for new therapies that leverage Type-A GABA neurotransmitter receptor function, particularly for the treatment of cancer and neurological disorders associated with GABA_{A} receptor function.

### SUMMARY

Accordingly, provided herein are benzodiazepine analogs that enhance chloride anion efflux in cancer cells, thereby initiating a cascade of events that impairs cancer cell viability. The disclosed benzodiazepine analogs have further application in the treatment of neurological disorders associated with GABA_{A} receptor function.

In one embodiment, a compound according to Formula I, or a pharmaceutically acceptable salt, racemate, or enantiomer thereof is provided:

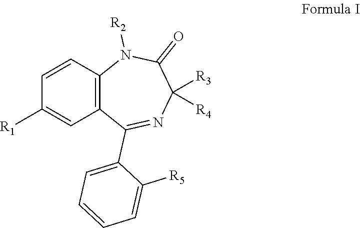

wherein R₁ is selected from the group consisting of C₂-C₄ alkynyl, C₃-C₆ cycloalkyl, d(5)-cyclopropyl, methyl alkynyl, alkynyl-CD₃, and alkynyl-CF₃; R₂ is selected from the group consisting of hydrogen, methyl, and trideuteromethyl; R₃ and R₄ are independently selected from the group consisting of hydrogen, deuterium, and methyl; and R₅ is selected from the group consisting of hydrogen, methyl, trideuteromethyl, tritritiomethyl, halogen, and trifluoromethyl. In embodiments, when R₂ is trideuteromethyl, R₁ is not ethynyl. In further embodiments, when R₁ is ethynyl, R₅ is not hydrogen.

In another embodiment, a pharmaceutical composition is provided, comprising: an effective amount of a compound according to Formula I; and a pharmaceutically acceptable carrier.

In another embodiment, a method of treating cancer in a subject in need thereof is provided, the method comprising administering to the subject an effective amount of a compound according to Formula I, or a pharmaceutically acceptable salt, racemate, or enantiomer thereof.

In another embodiment, a method of sensitizing a tumor to radiation in a subject in need thereof is provided, the method comprising administering to the subject an effective amount of a compound according to Formula I, or a pharmaceutically acceptable salt, racemate, or enantiomer thereof.

In another embodiment, a method of sensitizing a tumor to immunotherapy or chemotherapy in a subject in need thereof is provided, the method comprising administering to the subject an effective amount of a compound according to Formula I, or a pharmaceutically acceptable salt, racemate, or enantiomer thereof.

In another embodiment, a method of treating a neurological condition associated with Type-A GABA neurotransmitter receptor function in a subject in need thereof is provided, the method comprising administering to the subject an effective amount of a compound according to Formula I, or a pharmaceutically acceptable salt, racemate, or enantiomer thereof.

These and other objects, features, embodiments, and advantages will become apparent to those of ordinary skill in the art from a reading of the following detailed description and the appended claims.

### BRIEF DESCRIPTION OF THE DRAWINGS

The details of embodiments of the presently-disclosed subject matter are set forth in this document. Modifications to embodiments described in this document, and other embodiments, will be evident to those of ordinary skill in the art after a study of the information provided herein.

FIG. 1 depicts features of Type-A GABA receptors. GABA_{A} receptors move chloride anions across the extracellular plasma membrane when its agonist or ligand, GABA, binds (left panel). GABA_{A} receptors form pentameric assemblies. GABA_{A} receptor hetero-pentamers are composed of alpha, beta, and gamma subunits in alpha2-beta2-gamma1 stoichiometry (right panel). Benzodiazepines classically bind at the alpha-gamma interface of the GABA_{A} receptors and enhance the activity of GABA, thus commonly referred to as positive allosteric modulators.

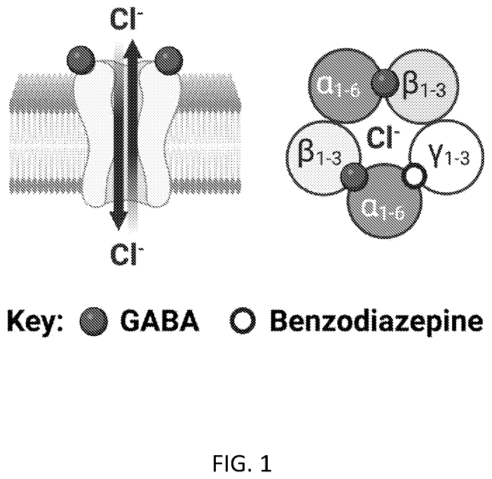

FIG. 2 depicts the common benzodiazepine structure according to the IUPAC numbering, which comprises a 1,4-diazepine ring system and a phenyl ring (left panel); the chemical structure of early benzodiazepine psychotropic drug diazepam (Valium) comprising 5-phenyl-1H-benzo[e][1,4]diazepin-2(3H)-one with a 7-chloro-1-methyl substituted (center panel); and the chemical structure of QH-II-066, comprising a 7-ethynyl in place of the 7-chloro on diazepam (right panel). This substitution contributes to its anti-cancer activity.

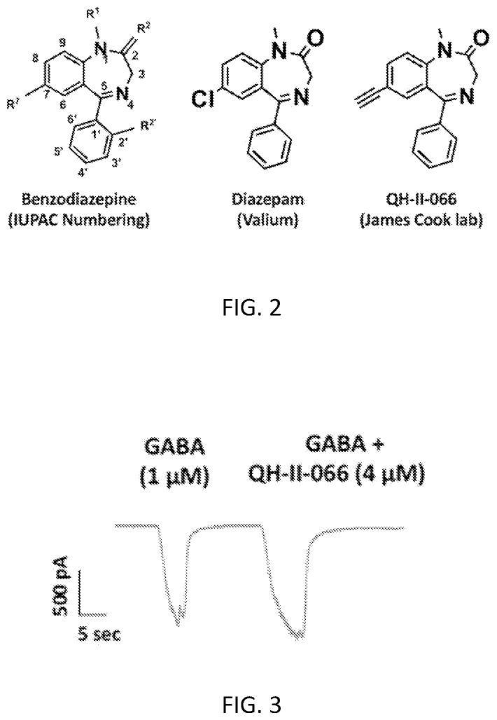

FIG. 3 depicts a representative trace of single-cell patch-clamp electrophysiology, wherein the response of patient-derived human lung cancer cell line H1792 to GABA (1 μM) and QH-II-066 (1 μM GABA+4 μM QH-II-066) is depicted, Notably, GABA+QH-II-066 enhances the current signal. Thus, this class of benzodiazepine analogs retain function as positive allosteric modulators of GABA_{A} receptors as well as possessing anti-cancer activity.

FIG. 4 depicts exemplary benzodiazepine analogs according to embodiments of the disclosure, grouped based on their substitution at the R7-position (IUPAC numbering) or R₁ of Formula I.

FIG. 5 depicts graphs demonstrating that benzodiazepine analogs depolarize cancer cells. An efflux of chloride anions induced by binding of the anti-cancer benzodiazepine analogs depolarizes the cancer cells. Shown are graphical representations of the binding of the cationic fluorescent dye TMRE (Tetramethylrhodamine, ethyl ester) to Lewis Lung Carcinoma cells monitored by Fluorescence-Activated Cell Sorting (FACS). Each of four graphs is of a different benzodiazepine analog: QH-II-066; and newly synthesized variants: TA-II-59, SRE-III-35, and SRE-III-43. Binding of the benzodiazepines causes a shift in electric charge distribution (depolarization) and thus reduced TMRE binding, as graphically reflected by a leftward shift. This occurs within 15 minutes of incubation with the benzodiazepine analogs. FCCP (2-[2-[4-(trifluoromethoxy)phenylhydrazinylidene]-propanedinitrile) is a potent mitochondrial oxidative phosphorylation uncoupler and serves as a positive control.

FIG. 6 is a summary of cytotoxicity (IC₅₀ values) of benzodiazepine analogs for different cancer cell lines (melanoma, A375; lung cancer, H1792; glioma, LN18; Lewis Lung Carcinoma (LLC)). NA, Not Active; *, Tested in vivo.

FIG. 7A depicts dose-response curves (graphs) showing cytotoxic effects of the R1-bromo benzodiazepine analogs TA-IV-08 on A375, H1792, LN18, and LLC cells; SRE-III-53 and SRE-III-54 on A375, H1792, and LN18 cells; TA-III-56 on H1792 cells. IC₅₀ values derived from these curves are reported in FIG. 6.

FIG. 7B depicts dose-response curves (graphs) showing cytotoxic effects of the R1-ethynyl benzodiazepine analogs QH-II-06 and TA-II-59, on A375, H1792, LN18, and LLC cells; TA-II-73 on A375, H1792, and LN18 cells; MYM-V-17, TA-III-50, TA-III-52, TA-III-62, TA-III-70, IT-04-75, and MYM-I-59 on H1792 cells. IC₅₀ values derived from these curves are reported in FIG. 6.

FIG. 7C depicts dose-response curves (graphs) showing cytotoxic effects of the R1-cyclopropyl benzodiazepine analogs SRE-III-35, SRE-III-43, TA-IV-74 on A375, H1792, LN18, and LLC cells; TA-IV-77 and TA-IV-87 on A375, H1792, and LN18 cells. IC₅₀ values derived from these curves are reported in FIG. 6.

FIG. 8 depicts the experimental set-up, test benzodiazepine analogs, and data obtained by dosing C57B16 (Black 6) immune-competent mice with bilateral flank Lewis Lung Carcinoma (LLC) tumors. As illustrated (top), mice were implanted with 5×10⁵ LLC cells in both left and right flanks. When flank tumors were palpable, mice received by intraperitoneal injection either vehicle control or one of four benzodiazepine analogs (structures shown) for seven consecutive days. There were N=3 mice per group and each mouse received benzodiazepine analog at 2.5 mg/Kg body weight per day for seven days. Tumor size was monitored over time and plotted relative to vehicle (control) mice (bottom). The most effective compound is TA-IV-74, a cyclopropyl benzodiazepine analog.

FIG. 9 are graphs depicting benzodiazepine analogs mediated enhanced formation of autophagosomes. Benzodiazepine analogs enhance formation of autophagosomes. Incubation of benzodiazepine analog with cancer cells (H1792) enhances puncta or autophagosomes relative to DMSO treated control cells. Puncta associated with key biomarkers LC3B (left) and NIX (right) are detected by confocal immunofluorescence microscopy and quantified per 3 cells, as shown here. LC3B: * p=0.02; Nix: * p=0.0377.

FIG. 10 are images of Western blots showing that benzodiazepine analogs enhance GABARAP and NIX protein production and multimerization. Left, Western blot analysis of GABARAP protein levels over time following incubation of lung cancer cells with a benzodiazepine analog. This shows an increased amount of GABARAP monomer (M) (see 72 hrs) and its dimer (D). Right, NIX protein levels also increase commensurate with increasing amount of benzodiazepine analog. NIX dimer (D) is evident at highest concentration shown. Dimerization and/or multimerization of these proteins are key to the initiation of autophagy.

FIG. 11 shows that an inhibitor of NIX binding to GABARAP abrogates benzodiazepine analog cytotoxicity. Combined treatment of H1792 cells with benzodiazepine analog and Pen-3-Ortho (IN), an inhibitory peptide that competes with NIX binding to GABARAP, abrogates cytotoxicity (left panel). Treatment of H1792 cells with IN reduces the stability of NIX monomer (M) and dimer (D) (right panel). This series of experiments illustrates that a critical part of the benzodiazepine mode-of-action is through GABARAP and NIX complex formation and their roles in autophagy. ** p<0.001; *** p<0.0001.

FIG. 12 is a schematic depicting the mode-of-action of benzodiazepine analog cytotoxicity. Cancer cells possess intrinsic receptor that mediates an efflux of chloride anions when GABA is bound (far left panel). When a benzodiazepine analog (BZ) binds to the receptor, chloride efflux is enhanced which depolarizes the mitochondrial transmembrane (second from left panel). This triggers enhanced expression of genes important to autophagy (third from left panel). The proteins GABARAP and NIX dimerize commensurate with multimerization of the receptor (fourth from left panel). This series of events triggers a further enhancement in receptor activity and formation of an autophagosome.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

For the purposes of defining the present technology, the transitional phrase “consisting of” may be introduced in the claims as a closed preamble term limiting the scope of the claims to the recited components or steps and any naturally occurring impurities. For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. The transitional phrases “consisting of” and “consisting essentially of” may be interpreted to be subsets of the open-ended transitional phrases, such as “comprising” and “including,” such that any use of an open ended phrase to introduce a recitation of a series of elements, components, materials, or steps should be interpreted to also disclose recitation of the series of elements, components, materials, or steps using the closed terms “consisting of” and “consisting essentially of.” For example, the recitation of a composition “comprising” components A, B, and C should be interpreted as also disclosing a composition “consisting of” components A, B, and C as well as a composition “consisting essentially of” components A, B, and C. Any quantitative value expressed in the present application may be considered to include open-ended embodiments consistent with the transitional phrases “comprising” or “including” as well as closed or partially closed embodiments consistent with the transitional phrases “consisting of” and “consisting essentially of.”

When the term “independently selected” is used, the substituents being referred to (e.g., R groups, such as groups R₃ and R₄), can be identical or different. For example, both R₃ and R₄ can be the same substituent, or R₃ and R₄ can each be different substituents selected from a specified group.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an,” and “the” include plural references unless the content clearly dictates otherwise.

A “pharmaceutically acceptable salt” is a cationic salt formed at any acidic (e.g., hydroxamic or carboxylic acid) group, or an anionic salt formed at any basic (e.g., amino) group. Many such salts are known in the art, as described in WO 1987/005297, by Johnston et al., published Sep. 11, 1987. Specific cationic salts include the alkali metal salts (such as sodium and potassium), and alkaline earth metal salts (such as magnesium and calcium) and organic salts. Specific anionic salts include halide (such as chloride, bromide, or fluoride salts), sulfate, and maleate. In embodiments, suitable pharmaceutically acceptable salts include, but are not limited to, halide, sodium, sulfate, acetate, phosphate, diphosphate, potassium, maleate, calcium, citrate, mesylate, nitrate, tartrate, aluminum, gluconate, carboxylate, and the like.

Such salts are well understood by the skilled artisan and the skilled artisan is able to prepare any number of salts given the knowledge in the art. Furthermore, it is recognized that the skilled artisan may select one salt over another for reasons of solubility, stability, formulation ease and the like. Determination and optimization of such salts is within the purview of the skilled artisan's practice.

The terms “enantiomer” and “racemate” have the standard art recognized meanings (see, e.g., Hawley's Condensed Chemical Dictionary, 16th ed. (2016)). The illustration of specific protected forms and other derivatives of the compounds of the instant invention is not intended to be limiting. The application of other useful protecting groups, salt forms, esters, and the like is within the purview of the skilled artisan.

The terms “halo” or “halogen,” as used herein, refer to fluoro (F), chloro (Cl), bromo (Br), and iodo (I) groups.

“Alkynyl,” as used herein, refers to a univalent hydrocarbon radical containing a triple bond. In embodiments, an alkynyl bond is represented as —C≡C. In embodiments, an alkynyl substituent is a C₂-C₄ alkynyl. In a specific embodiment, the alkynyl group is an ethynyl (also called ethinyl) group. Optionally, an alkynyl group may be substituted. Such substituted alkynyls include, but are not limited to, methyl alkynyl, alkynyl-D₃, alkynyl-CF₃, and the like.

“Cycloalkyl,” as used herein, refers to a C₃-C₆ inclusive hydrocarbon ring. Exemplary cycloalkyls include, but are not limited to, cyclopropyl, d(5)-cyclopropyl, cyclobutyl, cyclopentyl, cyclohexyl moieties. Optionally, a cycloalkyl group may be substituted with one or more short alkyls (C₁-C₆alkyl), deuterium, tritium, halogen, and the like.

D(5)-cyclopropyl refers to a cyclopropyl group wherein the hydrogen atoms are replaced with deuterium:

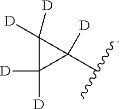 [SMILES: *C1([2H])C([2H])([2H])C1([2H])[2H]]

“Deuterium” (D), also known as heavy hydrogen or hydrogen-2, refers to an isotope of hydrogen that has one proton and one neutron in its nucleus and has twice the mass of hydrogen. A deuterated compound is a compound to which a deuterium atom has been introduced to replace hydrogen. Trideuteromethyl, or CD₃, is a methyl group wherein the hydrogen atoms have been replaced with deuterium.

“Tritium” (T), also known as hydrogen-3, refers to a radioactive isotope of hydrogen that has one proton and two neutrons. A tritiated compound is a compound to which a tritium atom has been introduced to replace hydrogen. Tritritiomethyl, or CT₃, is a methyl group wherein the hydrogen atoms have been replaced with tritium.

As used herein, the terms “treatment” or “treating” of a condition and/or a disease in an individual, including a human or lower mammal, means:
(i) preventing the condition or disease, that is, avoiding any clinical symptoms of the disease, particularly in individuals at risk for developing the condition or disease;
(ii) inhibiting the condition or disease, that is, arresting the development or progression of clinical symptoms; and/or
(iii) relieving the condition or disease, that is, causing the regression of clinical symptoms.

The terms “effective amount” or “therapeutically effective amount” as defined herein in relation to the treatment of cancer or neurological disorders, refer to an amount that will decrease, reduce, inhibit, or otherwise abrogate the growth of a cancer cell or tumor or arrest the development or progression of clinical symptoms of the neurological disorder. The specific therapeutically effective amount will vary with such factors as the particular disease being treated, the physical condition of the individual being treated, the duration of the treatment, the nature of concurrent therapy (if any), and the specific formulations employed.

As used herein, the terms “administer” or “administration” may comprise administration routes such as enteral (e.g., oral, sublingual, buccal, or rectal), parenteral (e.g., intravenous, intramuscular, subcutaneous, intraarterial, intratumoral), intranasal, inhaled, vaginal, transdermal, etc., so long as the route of administration results in an anti-cancer effect or treats the neurological disorder in the subject. In specific embodiments, the administration route is oral, intravenous, or intratumoral.

As used herein, the term “subject” generally refers to a living being (e.g., animal or human) capable of suffering from cancer or a neurological disorder. In a specific embodiment, the subject is a mammal. In a more specific embodiment, the subject is a human subject.

### Compounds

Provided herein are benzodiazepine analogs that enhance chloride anion efflux in cancer cells, thereby initiating a cascade of events that impairs cancer cell viability. The disclosed benzodiazepine analogs have further application in the treatment of neurological disorders associated with GABA_{A} receptor function.

The compounds disclosed herein are analogs of benzodiazepine compounds such as diazepam and QH-II-066, which compounds have the following structures:

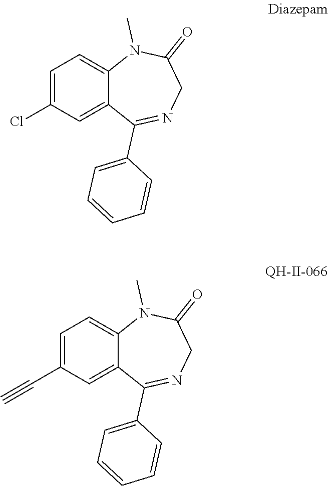 [SMILES: CN1C(=O)CN=C(c2ccccc2)c2cc(Cl)ccc21 ; C#Cc1ccc2c(c1)C(c1ccccc1)=NCC(=O)N2C]

In one embodiment, a compound according to Formula I is provided, or a pharmaceutically acceptable salt, racemate, or enantiomer thereof:

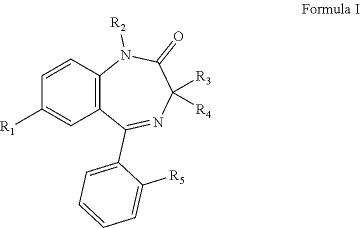

wherein R₁ is selected from the group consisting of C₂-C₄ alkynyl, C₃-C₆ cycloalkyl, d(5)-cyclopropyl, methyl alkynyl, alkynyl-CD₃, and alkynyl-CF₃; R₂ is selected from the group consisting of hydrogen, methyl, and trideuteromethyl; R₃ and R₄ are independently selected from the group consisting of hydrogen, deuterium, and methyl; and R₅ is selected from the group consisting of hydrogen, methyl, trideuteromethyl, tritritiomethyl, halogen, and trifluoromethyl. In certain embodiments, when R₂ is trideuteromethyl, R₁ is not ethynyl. In certain embodiments, when R₁ is ethynyl, R₅ is not hydrogen.

In some embodiments, R₃ and R₄ are each deuterium. In other embodiments, R₃ and R₄ are each hydrogen. In other embodiments, one or both of R₃ or R₄ is methyl.

In some embodiments, R₅ is a halogen selected from the group consisting of Cl, F, and Br.

In some embodiments, R₁ is cyclopropyl. In other embodiments, R₁ is ethynyl.

In a specific embodiment, R₁ is cyclopropyl or ethynyl; R₂ is hydrogen or methyl; R₃ and R₄ are each deuterium; and R₅ is selected from the group consisting of hydrogen, methyl, trideuteromethyl, tritritiomethyl, trifluoromethyl, fluorine, and chlorine.

In some embodiments, the compound is selected from the compounds set forth in Table 1:

TABLE 1
Exemplary Compounds

| | | | Formula/Molecular |
|---|---|---|---|
| Name | Chemical Name | Compound | Weight |
| TA-IV-08 | 7-bromo-1-methyl-5-(o- tolyl)-1,3-dihydro-2H- benzo[e][1,4]diazepin-2- one | 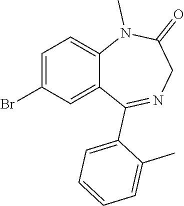 [SMILES: Cc1ccccc1C1=NCC(=O)N(C)c2ccc(Br)cc21] | C₁₇H₁₅BrN₂O/343.22 |
| SRE-III-53 | 7-bromo-5-(o-tolyl)-1,3- dihydro-2H-benzo[e][1,4] diazepin-2-one | 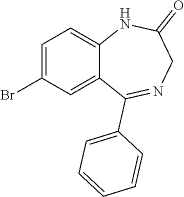 [SMILES: O=C1CN=C(c2ccccc2)c2cc(Br)ccc2N1] | C₁₅H₁₁BrN₂O/315.17 |
| SRE-III-54 | 7-bromo-5-(2- fluorophenyl)-1,3-dihydro- 2H-benzo[e][1,4]diazepin- 2-one | 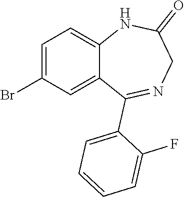 [SMILES: O=C1CN=C(c2ccccc2F)c2cc(Br)ccc2N1] | C₁₅H₁₀BrFN₂O/333.16 |
| TA-III-56 | 7,9-dibromo-5-phenyl-1,3- dihydro-2H- benzo[e][1,4]diazepin-2-one | 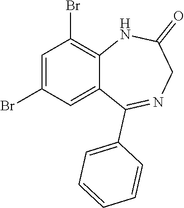 [SMILES: O=C1CN=C(c2ccccc2)c2cc(Br)cc(Br)c2N1] | C₁₅H₁₀Br₂N₂O/394.07 |
| TA-II-59 | 7-ethynyl-5-(o-tolyl)-1,3- dihydro-2H- benzo[e][1,4]diazepin-2-one | 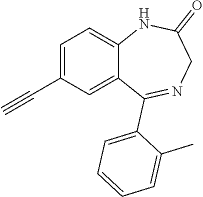 [SMILES: C#Cc1ccc2c(c1)C(c1ccccc1C)=NCC(=O)N2] | C₁₈H₁₄N₂O/274.32 |
| TA-II-73 | 7-ethynyl-1-methyl-5-(o- tolyl)-1,3-dihydro-2H- benzo[e][1,4]diazepin-2- one | 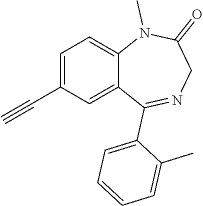 [SMILES: C#Cc1ccc2c(c1)C(c1ccccc1C)=NCC(=O)N2C] | C₁₉H₁₆N₂O/288.35 |
| TA-III-50 | 7-ethynyl-5-(2- fluorophenyl)-1-methyl-1,3- dihydro-2H- benzo[e][1,4]diazepine-2- thione | 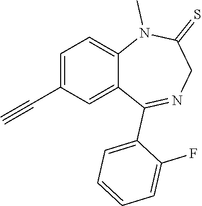 [SMILES: C#Cc1ccc2c(c1)C(c1ccccc1F)=NCC(=S)N2C] | C₁₈H₁₃FN₂S/308.37 |
| TA-III-52 | 7-ethynyl-1-methyl-5- phenyl-1,3-dihydro-2H- benzo[e][1,4]diazepine-2- thione | 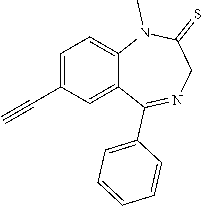 [SMILES: C#Cc1ccc2c(c1)C(c1ccccc1)=NCC(=S)N2C] | C₁₈H₁₄N₂S/290.38 |
| TA-III-62 | 7,9-diethynyl-1-methyl-5- phenyl-1,3-dihydro-2H- benzo[e][1,4]diazepin-2-one | 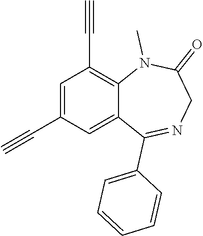 [SMILES: C#Cc1cc(C#C)c2c(c1)C(c1ccccc1)=NCC(=O)N2C] | C₁₈H₁₄N₂S/290.38 |
| TA-III-70 | 7-ethynyl-3-hydroxy-1- methyl-5-phenyl-1,3- dihydro-2H- benzo[e][1,4]diazepin-2-one | 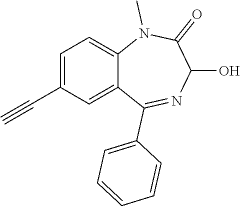 [SMILES: C#Cc1ccc2c(c1)C(c1ccccc1)=NC(O)C(=O)N2C] | C₁₈H₁₄N₂O₂/290.32 |
| MYM-I-59 | 7-ethynyl-5-(2- fluorophenyl)-3-hydroxy- 1,3-dihydro-2H- benzo[e][1,4]diazepin-2-one | 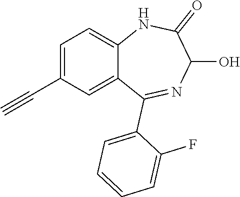 [SMILES: C#Cc1ccc2c(c1)C(c1ccccc1F)=NC(O)C(=O)N2] | C₁₇H₁₁FN₂O₂/294.29 |
| TI-04-75 | 8-ethynyl-6-(2- fluorophenyl)-1-methyl-4H- benzo[f]imidazo[1,5- a][1,4]diazepine | 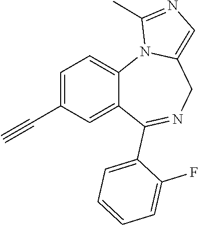 [SMILES: C#Cc1ccc2c(c1)C(c1ccccc1F)=NCc1cnc(C)n1-2] | C₂₀H₁₄FN₃/315.35 |
| MYM-V-17 | 5-(2-chlorophenyl)-7- ethynyl-3-hydroxy-1,3- dihydro-2H- benzo[e][1,4]diazepin-2-one | 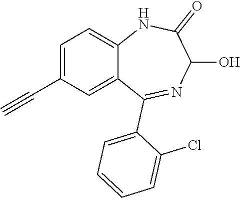 [SMILES: C#Cc1ccc2c(c1)C(c1ccccc1Cl)=NC(O)C(=O)N2] | C₁₇H₁₁ClN₂O₂/310.74 |
| TA-IV-74 | 7-cyclopropyl-1-methyl-5- phenyl-1,3-dihydro-2H- benzo[e][1,4]diazepin-2-one | 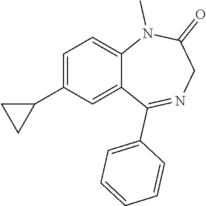 [SMILES: CN1C(=O)CN=C(c2ccccc2)c2cc(C3CC3)ccc21] | C₁₉H₁₈N₂O/290.37 |
| TA-IV-77 | 7-cyclopropyl-5-(2- fluorophenyl)-1-methyl-1,3- dihydro-2H- benzo[e][1,4]diazepin-2-one | 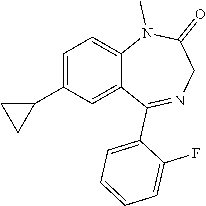 [SMILES: CN1C(=O)CN=C(c2ccccc2F)c2cc(C3CC3)ccc21] | C₁₉H₁₇FN₂O/308.36 |
| TA-IV-87 | 7-cyclopropyl-1-methyl-5- (o-tolyl)-1,3-dihydro-2H- benzo[e][1,4]diazepin-2-one | 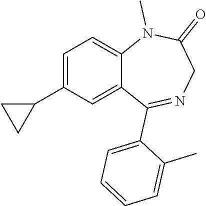 [SMILES: Cc1ccccc1C1=NCC(=O)N(C)c2ccc(C3CC3)cc21] | C₂₀H₂₀N₂O/304.39 |
| SRE-III-35 | 7-cyclopropyl-5-phenyl-1,3- dihydro-2H- benzo[e][1,4]diazepin-2-one | 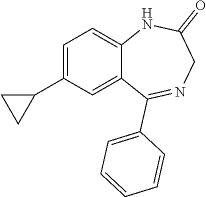 [SMILES: O=C1CN=C(c2ccccc2)c2cc(C3CC3)ccc2N1] | C₁₈H₁₆N₂O/276.34 |
| SRE-III-43 | 7-cyclopropyl-5-(2- fluorophenyl)-1,3-dihydro- 2H-benzo[e][1,4]diazepin- 2-one | 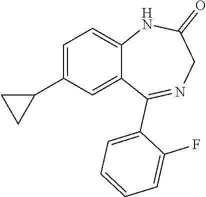 [SMILES: O=C1CN=C(c2ccccc2F)c2cc(C3CC3)ccc2N1] | C₁₈H₁₅FN₂O/294.33 |

### Exemplary Synthetic Routes

### Scheme 1: Preparation of TA-II-73

In embodiments, benzodiazepine analogs of the present disclosure differ from diazepam by a 2′ methyl and an ethynyl moiety in place of Cl.

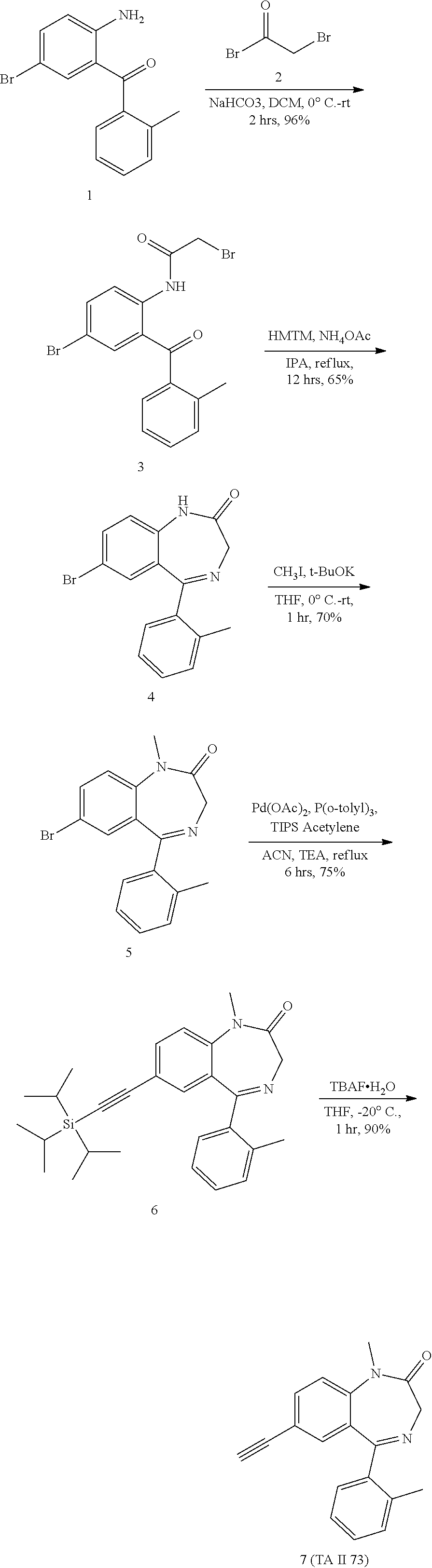

### Scheme 2: Preparation of N-H TA-II-59

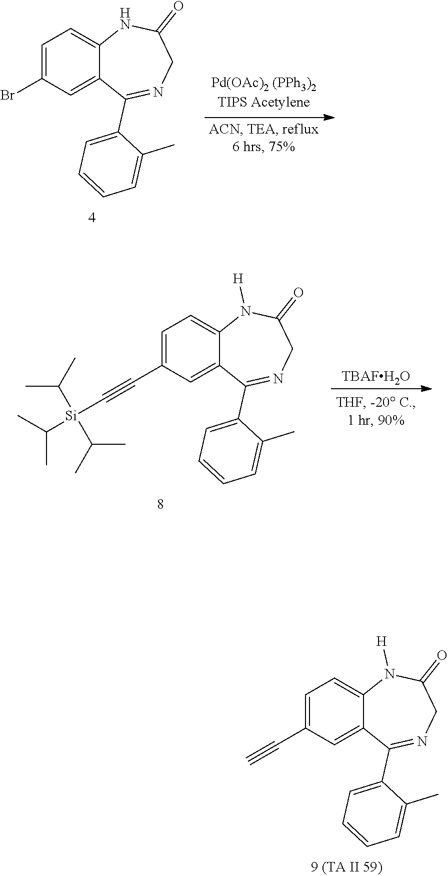 [SMILES: Cc1ccccc1C1=NCC(=O)Nc2ccc(Br)cc21 ; Cc1ccccc1C1=NCC(=O)Nc2ccc(C#C[Si](C(C)C)(C(C)C)C(C)C)cc21 ; "TBAF·H2O" ; C#Cc1ccc2c(c1)C(c1ccccc1C)=NCC(=O)N2]

### Scheme 3: Preparation of SRE-III-35 and SRE-III-43

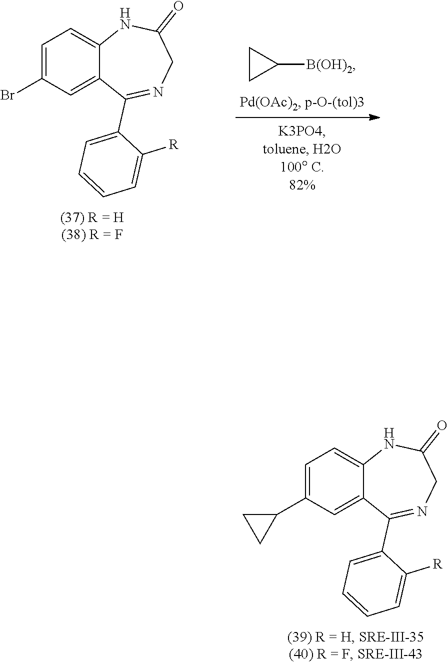

### 2-bromo-N-(4-bromo-2-(2-methylbenzoyl) phenyl) acetamide (3)

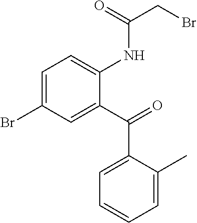 [SMILES: Cc1ccccc1C(=O)c1cc(Br)ccc1NC(=O)CBr]

To a mixture of (2-amino-5-bromophenyl) (o-tolyl) methanone 1 (10 g, 34.46 mmol), sodium bicarbonate (5.79 g, 68.92 mmol), and dichloromethane (100 mL), bromoacetyl bromide (2) (3.60 mL, 41.35 mmol) was added dropwise. The temperature was kept between −10° C.-0° C. with continuous stirring. The white colored reaction mixture, which resulted, was then allowed to stir for longer than 3 h at room temperature (rt). The completion of the reaction was verified by analysis by TLC (silica gel) and 50% ethyl acetate/hexanes. The reaction mixture was then slowly diluted over 30 min with water (100 mL) as carbon dioxide bubbles occurred. The biphasic mixture, which resulted, was allowed to stand for 15 min and the layers were separated. The aq layer was extracted with dichloromethane (100 mL) and the combined organic layers were washed with 5% aq sodium bicarbonate solution (100 mL) and then 10% aq sodium chloride solution (300 mL). The organic layer was dried (Na₂SO₄). The solvents were removed under reduced pressure and the residue was slurried with ethanol (100 mL) at 50-55° C. for 30 min. Upon cooling to rt and after holding the temperature for 1 h, the solid, which formed, was filtered, and washed with ethanol (60 mL×3). The solid was dried under vacuum at 40° C. to afford the product 2-bromo-N-(4-bromo-2-(2-methylbenzoyl) phenyl) acetamide 2 as an off-white solid (3) (13.56 g, 95.5%).

¹H NMR (300 MHz, CDCl₃) δ 12.01 (s, 1H), 8.66 (d, J=9.0 Hz, 1H), 7.71 (d, J=9.0 Hz, 1H), 7.55 (s, 1H), 7.44 (s, 1H), 7.35 (s, 2H), 4.07 (s, 2H), 2.35 (s, 3H). ¹³C NMR (300 MHz, CDCl₃) δ 190.64 (s), 172.73 (s), 139.27 (s), 138.47 (s), 137.96 (s), 131.86 (s), 130.16 (s), 129.35 (s), 128.66 (s), 127.56 (s), 125.65 (s). 124.35 (s), 122.76 (s), 36.02 (s), 19.82 (s); HRMS (ESI/IT-TOF) m/z: [M+H]+ Calcd for C₁₆H₁₃BrNO₂ 411.0869. found 411.0851.

### 7-bromo-5-(o-tolyl)-1,3-dihydro-2H-benzo[e][1,4]diazepin-2-one (4)

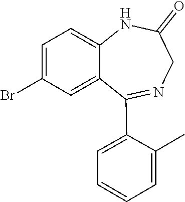 [SMILES: Cc1ccccc1C1=NCC(=O)Nc2ccc(Br)cc21]

A mixture of 2-bromo-N-(4-bromo-2-(2-methylbenzoyl) phenyl) acetamide, 3 (10 g, 24.3 mmol), hexamethylenetetramine (HMTM, 7.50 g, 53.5 mmol), ammonium acetate (4.12 g, 53.5 mmol), and isopropanol (100 mL) was heated to reflux (82° C.). The reaction mixture was held at reflux for 6 h, at which point the reaction progress was deemed complete on analysis by TLC (silica gel and 1:1, ethyl acetate/hexanes). The reaction mixture was then cooled to 0-5° C. using an ice bath. The solid, which resulted, was filtered, and washed with cold isopropanol (100 mL×2) and then water (100 mL×4). The solid was dried under vacuum at 40° C. to afford 3.2 g of the benzodiazepine 72 as an off-white solid. The IPA was removed from the mother liquor under reduced pressure. The solid was then extracted with ethyl acetate. The ethyl acetate was removed under reduced pressure and the residue was purified by column chromatography using 1:4 ethyl acetate/hexanes to afford 2.01 grams more of 7-bromo-5-(o-tolyl)-1,3-dihydro-2H-benzo[e][1,4]diazepin-2-one (4) (5.21 g, 65%).

¹H NMR (300 MHz, CDCl₃) δ 11.95 (s, 1H), 8.68 (d, J=9.0 Hz, 1H), 7.65 (d, J=9.0 Hz, 1H), 7.45 (d, J=22.1 Hz, 2H), 7.27-7.17 (m, 2H), 7.14 (d, J=7.6 Hz, 1H), 3.65 (s, 2H), 2.23 (s, 3H). ¹³C NMR (75 MHz, CDCl₃) δ 200.64, 170.52, 139.35, 138.24, 137.51, 136.52, 136.16, 131.20, 130.77, 128.31, 125.50, 122.95, 115.09, 53.65, 19.80. HRMS (ESI/IT-TOF) m/z: [M+H]+ Calcd for C₁₇H₁₅BrN₂O, 329.1892. found 329.1922.

### 7-bromo-1-methyl-5-(o-tolyl)-1,3-dihydro-2H-benzo[e] [1,4] diazepin-2-one (5)

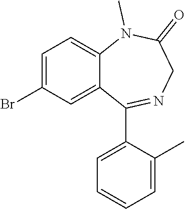 [SMILES: Cc1ccccc1C1=NCC(=O)N(C)c2ccc(Br)cc21]

The 7-bromo-5-(o-tolyl)-1,3-dihydro-2H-benzo[e][1,4]diazepin-2-one 4, (5.0 g, 15.2 mmol) was dissolved in THF, (30 mL) and the solution was cooled to −0° C. using an ice bath. Then potassium tert butoxide (1.87 g, 16.7 mmol) which was dissolved in 30 mL of THF was added dropwise by using an addition funnel. Then methyl iodide (1.14 mL, 18.2 mmoL) was added dropwise to the reaction mixture over a 1 min period, while maintaining the temperature at 0° C. Upon completion of the addition, the reaction mixture was allowed to warm to rt and stir for 60 min, at which point the reaction was deemed complete on analysis by TLC (silica gel). The reaction mixture was then diluted with ethyl acetate (20 mL) and a solution of 10% aq sodium chloride (200 mL) was added. The biphasic mixture, which resulted, was allowed to stand for 15 min and the layers were separated. The aq layer was then extracted with ethyl acetate (50 mL) and the combined organic layers were washed with 10% aq sodium chloride solution (50 mL). The organic layer was dried (Na₂SO₄). The solvent was removed under reduced pressure. The brown solid which was obtained was purified by crystallization using 15:85 (ethyl acetate/hexanes), to give 7-bromo-1-methyl-5-(o-tolyl)-1,3-dihydro-2H-benzo[e][1,4]diazepin-2-one (5) (3.91 g, 75%) as a brownish white solid.

¹H NMR (300 MHz, CDCl₃) δ 7.42 (d, J=6.4 Hz, 4H), 7.28 (d, J=4.1 Hz, 1H), 7.17 (d, J=8.1 Hz, 1H), 7.01 (d, J=2.2 Hz, 1H), 4.33 (s, 2H), 3.87 (s, 3H), 2.10 (s, 3H). ¹³C NMR (126 MHz, CDCl3) δ 169.93, 168.86, 143.10, 138.19, 134.37, 132.89, 130.73, 129.50, 128.45, 122.79, 116.82, 56.94, 34.85, 19.98. HRMS (ESI/IT-TOF) m/z: [M+H]+ Calcd for C₁₇H₁₅BrN₂O, 343.2169. found 343.2152.

### 1-methyl-5-(o-tolyl)-7-((triisopropylsilyl)ethynyl)-1,3-dihydro-2H-benzo[e] [1,4] diazepin-2-one (6)

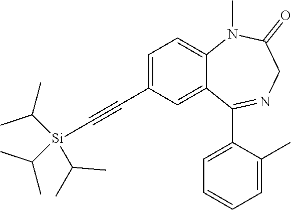 [SMILES: Cc1ccccc1C1=NCC(=O)N(C)c2ccc(C#C[Si](C(C)C)(C(C)C)C(C)C)cc21]

In a 500 mL round bottom flask, Pd(OAc)₂ (112 mg, 0.5 mmol) and P(o-tolyl)3 (304.37 mg, 1.0 mmol) was added to 50 mL of acetonitrile. The mixture was stirred until a slurry appeared, which took about 20 min. Then 7-bromo-1-methyl-5-phenyl-1,3-dihydro-2H-1,4-benzodiazepin-2-one 5, (3.50 g, 10.2 mmol), triethylamine (4.26 mL, 30.6 mmol), (triisopropylsilyl)acetylene (3.43 mL, 15.3 mmol) and additional acetonitrile (50 mL) was added. The reaction mixture was then heated to reflux (75° C.) and held for 6 h, at which point the reaction was deemed complete on analysis by TLC (Silica gel). Upon completion of the reaction progress, the mixture was cooled to rt and filtered through celite. After washing with acetonitrile (100 mL×2), the solvents were removed under reduced pressure and the residue was dissolved in dichloromethane (400 mL) and 5% aq sodium bicarbonate (400 mL) was added. The biphasic mixture, which resulted, was allowed to stand for 15 min and the layers were separated. The aq layer was then extracted with dichloromethane (300 mL) and the combined organic layers were washed with 5% aq sodium bicarbonate solution (300 mL) and then 10% aq sodium chloride solution (300 mL×3). The organic layer was dried (Na₂SO₄) and it was purified by flash chromatography on silica gel. This process gave a dark orange liquid that gets solidified eventually on standing to give 1-methyl-5-phenyl-7-((tripropan-2-ylsilyl) ethynyl)-1,3-dihydro-2H-1,4-benzodiazepin-2-one (6) (3.4, 71% crude yield).

¹H NMR (500 MHz, CDCl₃) δ7.24-7.52 (m, 7H), 3.48 (s, 2H), 3.33 (s, 3H), 2.21 (s, 3H), 1.27 (m, 3H), 1.0 (d, 18H); ¹³C NMR (500 MHz, CDCl3) δ 177.23 (s), 168.21 (s), 137.86 (s), 132.36 (s), 129.35 (s), 128.97 (s), 128.56 (s), 128.37 (s), 125.65 (s), 122.47 (s), 92.37 (s), 91.53 (s), 43.84 (s), 33.82 (s), 19.82 (s), 15.53 (s, 3C), 9.43 (s, 6C).

### 7-ethynyl-1-methyl-5-(o-tolyl)-1,3-dihydro-2H-benzo[e] [1,4] diazepin-2-one (7)

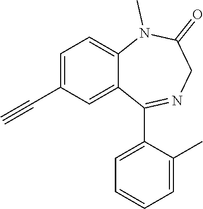 [SMILES: C#Cc1ccc2c(c1)C(c1ccccc1C)=NCC(=O)N2C]

1-Methyl-5-phenyl-7-((tripropan-2-ylsilyl) ethynyl)-1,3-dihydro-2H-1,4-benzodiazepin-2-one 6, (3.40 g, 7.6 mmol), water (0.5 mL) and tetrahydrofuran (30 mL) were cooled to −20° C. using a dry ice/IPA bath. Then tetrabutylammonium fluoride hydrate, [1 M in THF (10.9 mL, 10.9 mmoL)] was added dropwise to the reaction mixture over a 30 min period, while maintaining the temperature at −20 to −15° C. Upon completion of the addition, the reaction mixture was allowed to warm to rt and stir for an additional 60 min at which point the reaction progress was deemed complete on analysis by TLC (silica gel). The reaction mixture was then diluted with ethyl acetate (50 mL) and 10% aq sodium chloride (50 mL). The biphasic mixture, which resulted, was allowed to stand for 15 min and the layers were separated. The aq layer was then extracted with ethyl acetate (50 mL×3) and the combined organic layers were washed with 10% aq sodium chloride solution (150 mL). The organic layer was dried (Na₂SO₄). The solvents were removed under reduced pressure. Then the mixture was dissolved in 50 mL of ethyl acetate and then stirred with 20 g of silica gel for 2 hours and filtered. The amount of solvent was reduced to about 40 mL under reduced pressure. Then 20 mL of hexanes was added dropwise to the mixture, and it was allowed to stir overnight. The solid, which formed, was filtered and the grey solid was recrystallized from 1:4 (ethyl acetate/hexanes) to obtain cream white colored TA-II-73 (7). (1.98 g, 90%).

¹H NMR (500 MHz, CDCl₃) δ 7.63-7.60 (m, 1H), 7.38-7.27 (m, 4H), 7.24-7.18 (m, 2H), 4.86 (t, J=11.4 Hz, 1H), 3.81 (dd, J=18.3, 10.8 Hz, 1H), 3.46 (s, 3H), 3.05 (s, 1H), 1.99 (d, J=8.0 Hz, 3H). ¹³C NMR (126 MHz, CDCl₃) δ 171.32, 169.86, 143.10, 138.91, 136.26, 134.67, 133.12, 130.84, 130.49, 129.67, 128.39, 125.97, 121.12, 118.19, 81.95, 78.27, 56.79, 34.72, 19.94. HRMS (ESI/IT-TOF) m/z: [M+H]+ Calcd for C₁₉H₁₆N₂O, 288.3420. found 288.3406.

### 7-cyclopropyl-5-phenyl-1,3-dihydro-2H-benzo[e][1,4]diazepin-2-one (39)

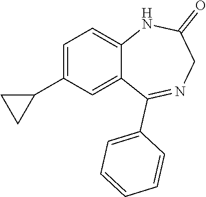 [SMILES: O=C1CN=C(c2ccccc2)c2cc(C3CC3)ccc2N1]

To a solution of palladium (II) acetate (0.099 g, 0.44 mmol) and tri-o-tolyl phosphine (0.27 g, 0.88 mmol) in toluene, the 7-bromo-5-phenyl-1,3-dihydro-2H-benzo[e][1,4]diazepin-2-one (2.0 g, 6.3 mmol) was added. Then in sequence, cyclopropyl boronic acid (2.45 g, 28.52 mmol), and bis(triphenylphosphine) (5.38 g, 25.37 mmol) and water (0.5 mL) were added under argon. A reflux condenser was attached, and the reaction mixture was heated to reflux. The mixture was stirred and heated to 100° C. After 12 h the reaction was completed on analysis by mass spectroscopy, and it was then cooled to rt and silica gel (3 g) was added. After stirring for 30 minutes open to the air, the spent catalyst on silica gel was removed by filtration through a pad of celite and washed with EtOAc. Then the filtrate was concentrated under reduced pressure. The residue which resulted was purified by a wash column (silica gel, EtOAc) to afford the desired product as a white solid (39) (1.29 g, 74%).

¹H NMR (500 MHz, CDCl₃) δ 9.67 (s, 1H), 7.65 (d, J=7.5 Hz, 1H), 7.55 (dd, J=14.8, 7.3 Hz, 2H), 7.47 (t, J=7.5 Hz, 2H), 7.25 (d, J=7.6 Hz, 1H), 7.21 (d, J=8.0 Hz, 1H), 7.02 (d, J=1.7 Hz, 1H), 4.29 (s, 2H), 1.89-1.82 (m, 1H), 0.98-0.94 (m, 2H), 0.64-0.57 (m, 2H). ¹³C NMR (126 MHz, CDCl₃) δ170.3, 166.00, 139.66, 138.35, 137.24, 132.71, 131.34, 130.98, 130.68, 129.95, 129.57, 129.22, 121.85, 121.69, 47.10, 14.83, 9.32, 9.18.

### 7-cyclopropyl-5-(2-fluorophenyl)-1,3-dihydro-2H-benzo[e][1,4]diazepin-2-one (40)

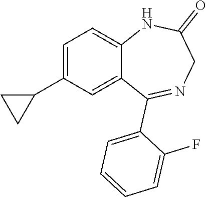 [SMILES: O=C1CN=C(c2ccccc2F)c2cc(C3CC3)ccc2N1]

To a solution of palladium (II) acetate (0.07 g, 0.31 mmol) and tri-o-tolyl phosphine (0.19 g, 0.63 mmol) in toluene, the 7-bromo-5-(2-fluorophenyl)-1,3-dihydro-2H-benzo[e][1,4]diazepin-2-one (1.5 g, 4.5 mmol) was added. Then in sequence, cyclopropyl boronic acid (1.74 g, 20.25 mmol), and bis(triphenylphosphine) (3.81 g, 18 mmol) and water (0.36 mL) were added under argon. A reflux condenser was attached, and the reaction mixture was heated to reflux. The mixture was stirred and heated to 100° C. After 12 h the reaction was completed on analysis by mass spectroscopy, and it was then cooled to rt and silica gel (2.5 g) was added. After stirring for 30 minutes open to the air, the spent catalyst on silica gel was removed by filtration through a pad of celite and washed with EtOAc. Then the filtrate was concentrated under reduced pressure. The residue which resulted was purified by a wash column (silica gel, EtOAc) to afford the desired product as a white solid (40) (0.99 g, 75%).

¹H NMR (500 MHz, CDCl₃) δ 9.66 (s, 1H), 7.62 (t, J=7.2 Hz, 1H), 7.57 (t, J=7.4 Hz, 1H), 7.31 (d, J=8.2 Hz, 1H), 7.29 (s, 1H), 7.26 (d, J=7.1 Hz, 1H), 7.24 (s, 1H), 6.95 (s, 1H), 4.36 (s, 2H), 1.86-1.80 (m, 1H), 0.94 (dt, J=6.3, 3.1 Hz, 2H), 0.59 (dt, J=10.0, 4.9 Hz, 2H). ¹³C NMR (126 MHz, CDCl₃) δ 170.2, 165.27, 161.59, 159.57, 140.13, 139.0, 134.6, 132.61, 132.14, 124.40, 123.95, 121.72, 116.46, 116.40, 116.23, 14.80, 9.25, 9.05.

### Synthesis of N-CD₃ Analogs of QH-II-066 and KRM-II-08

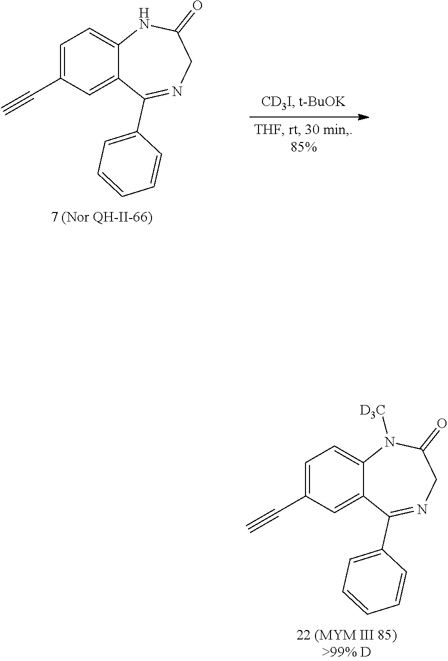 [SMILES: C#Cc1ccc2c(c1)C(c1ccccc1)=NCC(=O)N2 ; [2H]C([2H])([2H])N1C(=O)CN=C(c2ccccc2)c2cc(C#C)ccc21]

Two N-CD₃ compounds were synthesized designated TA-I-16 and MYM-III-85. TA-I-16 can be synthesized from NOR KRM-II-08 by treating the amide with deuterated methyl iodide in the presence of potassium tert-butoxide in THF at 0° C. to room temperature. MYM-III-85 can be synthesized from nor QH-II-066 via a similar route. The final products can be purified by column chromatography with ethyl acetate-hexanes (20:80). KRM-II-08 has the following structure:

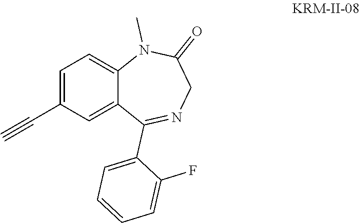 [SMILES: C#Cc1ccc2c(c1)C(c1ccccc1F)=NCC(=O)N2C]

### Synthesis of D₂-QH-II-066

To synthesize D₂-QH-II-066, one must o exchange the hydrogen atoms at the C-3 position of QH-II-066. To do this, a sufficiently strong base and a deuterated solvent are needed. 50 mg of QH-II-066 are dissolved in in 1 mL of D4-methanol. 1 equivalent of base is added and the mixture is stirred for 1 h at room temperature. The precipitate is then filtered using a PTFE filter. The D4-methanol is evaporated on a rotary evaporator under reduced pressure. Exchange of H for D is confirmed via ¹H NMR. The solid is dissolved in regular methanol and the solvent is evaporated 5-6 times to regenerate the acetylene hydrogen atom.

Potassium tert-butoxide is a suitable strong base. Applying heat after the workup increases the percent of deuterium exchange. Without heat, one cannot regenerate back the acetylene hydrogen. 95+% deuterium is preferred for use in in vivo assays.

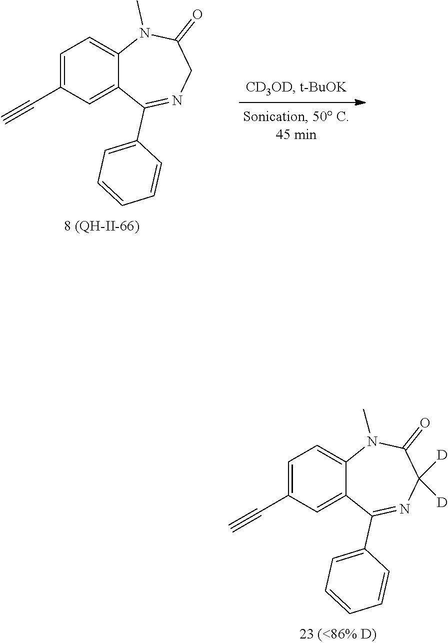 [SMILES: C#Cc1ccc2c(c1)C(c1ccccc1)=NCC(=O)N2C ; [2H]C1([2H])N=C(c2ccccc2)c2cc(C#C)ccc2N(C)C1=O]

### Synthesis of D₅-QH-II-066

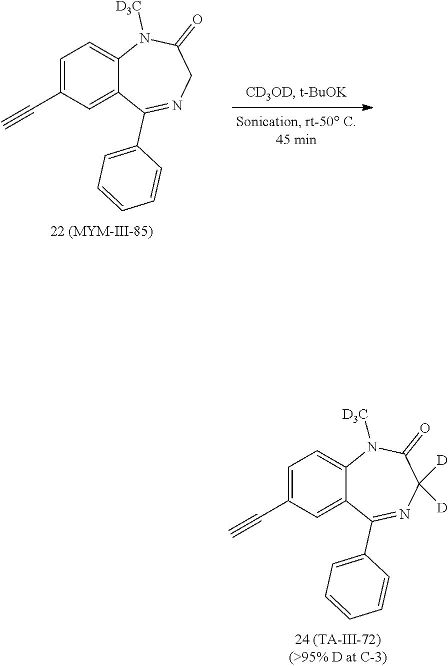 [SMILES: [2H]C([2H])([2H])N1C(=O)CN=C(c2ccccc2)c2cc(C#C)ccc21 ; [2H]C1([2H])N=C(c2ccccc2)c2cc(C#C)ccc2N(C([2H])([2H])[2H])C1=O]

D₃-MYM-III-85 was used as a starting material. This compound contains an N-CD₃ moiety. A similar procedure was followed as was done for D₂-QH-II-066 for the deuteration of MYM-III-85, but the results were the same as observed in the case of D₂-QH-II-066. Potassium tert-butoxide was used as the base and D4-methanol was used as the solvent.

MYM-III-85 was dissolved in D4-methanol and 1.1 equivalents of potassium tert-butoxide was added. The mixture was sonicated at 50° C. for one hour. Then, 4 mL of DI water were added to the mixture at room temperature. This solution was extracted with ethyl acetate and the solvent was removed under an argon flow. After this, a 96% deuterium exchange at the C-3 position of MYM-III-85 was observed, providing 95%+D₅-QH-II-066, which was assigned code number of TA-III-72.

### 7-ethynyl-1-(methyl-d3)-5-phenyl-1,3-dihydro-2H-benzo[e][1,4]diazepin-2-one. (22)

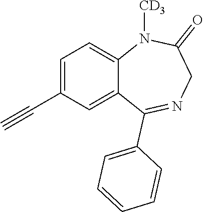 [SMILES: [2H]C([2H])([2H])N1C(=O)CN=C(c2ccccc2)c2cc(C#C)ccc21]

The 5-phenyl-7-((ethynyl)-1,3-dihydro-2H-1,4-benzodiazepin-2-one (7), (4.0 g, 15.34 mmol) was dissolved in THF (15 mL) and the solution was cooled to 0° C. using an ice bath. Then potassium tert butoxide (1.90 g, 16.92 mmol), which was dissolved in 20 mL of THF, was added dropwise by using an addition funnel. Then trideutero methyl iodide (1.24 mL, 19.94 mmoL) was added dropwise to the reaction mixture over a 5 min period, while maintaining the temperature at 0° C. Upon completion of the addition, the reaction mixture was allowed to warm to rt and stirred for 60 min, at which point the reaction was deemed complete on analysis by TLC (silica gel). The reaction mixture was then diluted with ethyl acetate (20 mL) and a solution of 10% aq sodium chloride solution (20 mL) was added. The biphasic mixture, which resulted, was allowed to stand for 15 min and the layers were separated. The aq layer was then extracted with ethyl acetate (20 mL) and the combined organic layers were washed with 10% aq sodium chloride solution (20 mL). The organic layer was dried (Na₂SO₄). The solvent was removed under reduced pressure. The brown solid, which was obtained, was purified by crystallization using 15:85 (ethyl acetate/hexanes), to give 7-ethynyl-1-(methyl-d3)-5-phenyl-1,3-dihydro-2H-benzo[e][1,4]diazepin-2-one (22)(3.7 g, 86%) as a white solid.

¹H NMR (500 MHz, CDCl₃): 7.82 (d, 1H) 7.66-7.64 (m, 2H, 2×ArH), 7.60-7.57 (m, 2H, 2×ArH), 7.52-7.49 (m, 2H, 2×ArH), 7.38 (dd, 1H, J=8.5, 2.0 Hz, H-4), 4.6 (D, 1H), 3.6 (D, 1H), 3.1 (s, 1H). ¹³C NMR (500 MHz, CDCl₃): 171.2, 168.21, 149.4, 139.5, 134.4, 133.4, 131.7, 129.3, 128.5, 120.3, 119.2, 118.8, 82.3, 79.2, 56.2, 34.2. Rf=0.5 (silica gel, ethyl acetate/hexanes 1:2) HRMS (ESI/IT-TOF): m/z [M+H]+ calcd for C₁₈H₁₁D₃N₂O: 277.3350. found 277.3329.

### 7-Ethynyl-1-(methyl-d3)-5-phenyl-1,3-dihydro-2H-benzo[e] [1,4] diazepin-2-one-3,3-d2 (24)

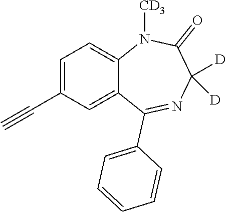 [SMILES: [2H]C1([2H])N=C(c2ccccc2)c2cc(C#C)ccc2N(C([2H])([2H])[2H])C1=O]

The 7-ethynyl-1-(methyl-d3)-5-phenyl-1,3-dihydro-2H-benzo[e][1,4]diazepin-2-one (22), (0.5 g, 1.8 mmol) was dissolved in D4-methanol (1 mL). Then potassium tert butoxide (0.22 g, 1.9 mmol) was added to the solution. The mixture was sonicated for 45 min starting from rt to 50° C. After the reaction was completed (checked by NMR) 5 ml of de-ionized water was added and the mixture was extracted with (5 mL×2) ethyl acetate. It was dried by first with blowing argon over it and then placing it in high vacuum. This gave 1-CD3-methyl-5-phenyl-7-ethynyl-1,3-dihydro-2d-1,4-benzodiazepin-2-one with 97% deuteration at the C-3 position via integration by NMR spectroscopy, to give 7-ethynyl-1-(methyl-d3)-5-phenyl-1,3-dihydro-2H-benzo[e][1,4]diazepin-2-one-3,3-d2 (D5-QH-II-066) (24) (0.49 g, 97%) as a white solid.

¹H NMR (500 MHz, CDCl₃): 7.82 (d, 1H) 7.66-7.64 (m, 2H, 2×ArH), 7.60-7.57 (m, 2H, 2×ArH), 7.52-7.49 (m, 2H, 2×ArH), 7.38 (dd, 1H, J=8.5, 2.0 Hz, H-4), 3.1 (s, 1H). ¹³C NMR (500 MHz, CDCl₃): 171.2, 168.21, 149.4, 139.5, 134.4, 133.4, 131.7, 129.3, 128.5, 120.3, 119.2, 118.8, 82.3, 79.2, 56.2, 34.2. Rf=0.5 (silica gel, ethyl acetate/hexanes 1:2), HRMS (ESI/IT-TOF): m/z [M+H]+ calcd for C₁₈H₉D₅N₂O: 279.3473. found 279.3422.

### Synthesis of Acetylene Analogs

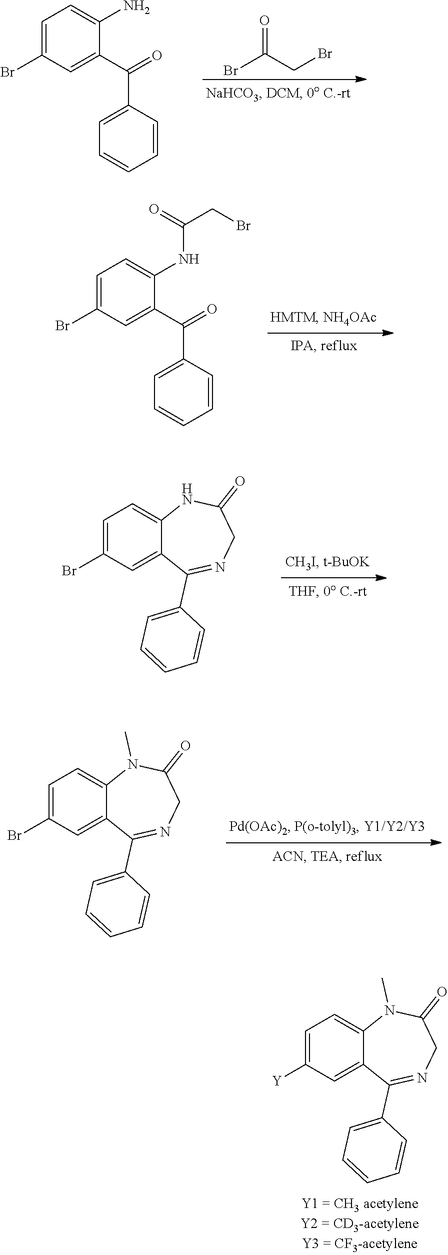

Synthesis of d(5)-Cyclopropyl Analogs

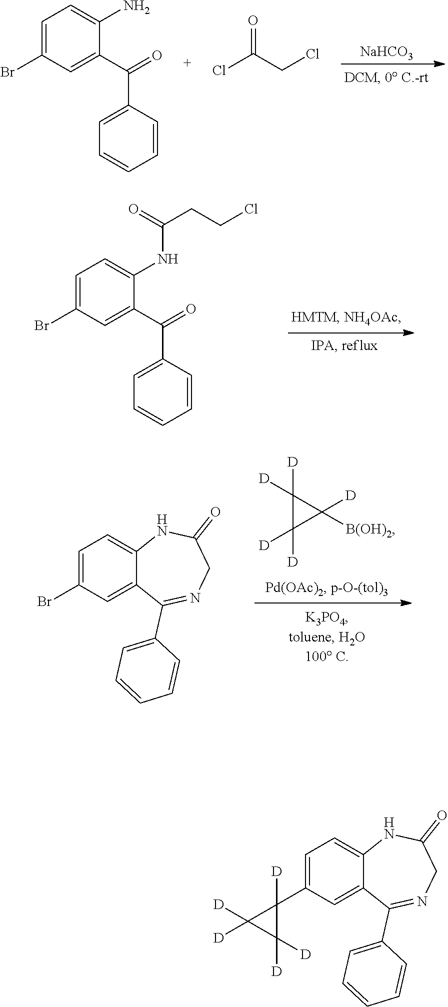

### Synthesis of Substituted Cycloalkyl Analogs

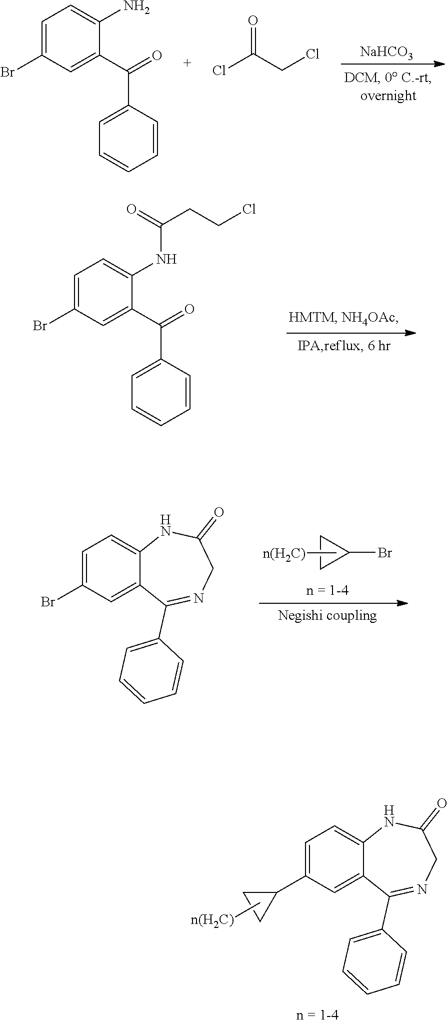

### Pharmaceutical Compositions

In another embodiment, a pharmaceutical composition is provided, the composition comprising a compound according to Formula I, or a pharmaceutically acceptable salt, racemate, enantiomer, or derivative thereof; and at least one pharmaceutically acceptable carrier. In embodiments, the pharmaceutical compositions disclosed herein are formulated for the treatment of cancer. In embodiments, the pharmaceutical compositions disclosed herein are formulated for the treatment of a neurological disorder associated with GABA_{A} receptor function.

The pharmaceutically acceptable excipient, or carrier, must be “acceptable” in the sense of being compatible with the other ingredients of the composition and not deleterious to the recipients thereof. The disclosure further includes a pharmaceutical composition, in combination with packaging material suitable for the pharmaceutical composition, including instructions for the use of the composition in the treatment of subjects in need thereof.

Pharmaceutical compositions include those suitable for enteral (e.g., oral, sublingual, buccal, or rectal), parenteral (e.g., intravenous, intramuscular, subcutaneous, intraarterial, intratumoral), intranasal, inhaled, vaginal, or transdermal administration. In a specific embodiment, the pharmaceutical compositions are formulated for intravenous administration, e.g., by injection or infusion. In another specific embodiment, the pharmaceutical compositions are formulated for oral administration.

The pharmaceutical compositions may be prepared by any methods well known in the art of pharmacy, for example, using methods such as those described in Remington: The Science and Practice of Pharmacy (21st ed., Lippincott Williams and Wilkins, 2005, see Part 5: Pharmaceutical Manufacturing). Suitable pharmaceutical carriers are well-known in the art. See, for example, Handbook of Pharmaceutical Excipients, Sixth Edition, edited by Raymond C. Rowe (2009). The skilled artisan will appreciate that certain carriers may be more desirable or suitable for certain modes of administration of an active ingredient. It is within the purview of the skilled artisan to select the appropriate carriers for a given composition.

For parenteral administration, suitable compositions include aqueous and non-aqueous sterile suspensions for intravenous administration. The compositions may be presented in unit dose or multi-dose containers, for example, sealed vials and ampoules.

For oral administration, suitable compositions include liquids, capsules, tablets, chewable tablets, soluble films, powders, and the like.

As will be understood by those of skill in this art, the specific dose level for any particular subject will depend on a variety of factors, including the activity of the agent employed; the age, body weight, general health, and sex of the individual being treated; the particular disease to be treated; the time and route of administration; the rate of excretion; and the like.

In embodiments, an effective dose of a Formula I compound according to the present disclosure may range from about 0.01 mg/kg/day to about 100 mg/kg/day, or from about 0.01 mg/kg/day to about 10 mg/kg/day, or from about 0.1 mg/kg/day to about 100 mg/kg/day, or from about 0.1 mg/kg/day to about 10 mg/kg/day, or from about 1 mg/kg/day to about 10 mg/kg/day. In embodiments, the dose of a Formula 1 compound is at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 30, 40, 50, 60, 70, 80, 90, or 100 mg/kg/day, or any selected range of values there between.

### Methods of Use

In another embodiment, a method of treating cancer in a subject in need thereof is provided, the method comprising administering to the subject an effective amount of a compound according to Formula I as disclosed herein, or a pharmaceutically acceptable salt, racemate, or enantiomer thereof.

In embodiments, the subject is a mammal. In a more specific embodiment, the subject is a human.

In embodiments, the cancer is any primary or metastatic solid tumor, including pediatric and adult tumors. In specific embodiments, the cancer is selected from the group consisting of melanoma, glioblastoma, medulloblastoma, neuroblastoma, and lung cancer. In a more specific embodiment, the lung cancer is non-small cell lung cancer (NSCLC).

In embodiments, administering comprises enteral or parenteral administration. In more specific embodiments, enteral administration comprises oral, sublingual, or buccal administration. In other specific embodiments, parenteral administration comprises intravenous, intramuscular, subcutaneous, intraarterial, or intratumoral administration. Compositions comprising Formula I compounds can be formulated for administration by any suitable enteral or parenteral administration.

In embodiments, the compound is administered at a dose of from about 0.1 mg/kg/day to about 100 mg/kg/day. In a more specific embodiment, the compound is administered at a dose of from about 1 mg/kg/day to about 30 mg/kg/day.

In embodiments, the methods disclosed herein further comprise administering to the subject one or more additional active agents. Illustratively, the one or more additional active agents are selected from the group consisting of an anti-inflammatory agent, an immunosuppressive agent, a corticosteroid, and a chemotherapeutic agent selected from the group consisting of an alkylating agent, a platinum drug, an antimetabolite, an anti-tumor antibiotic, a topoisomerase inhibitor, a mitotic inhibitor, a differentiating agent, an immune checkpoint inhibitor, and a hormone therapy.

In other embodiments, the methods disclosed herein further comprise administering radiation therapy to the subject. In embodiments, the methods disclosed herein further comprise administration of an immune checkpoint inhibitor, including but not limited to PD-1 inhibitors (e.g., pembrolizumab, nivolumab, cemiplimab, etc.); PD-L1 inhibitors (e.g., atezolizumab, avelumab, durvalumab, etc.); CTLA-4 inhibitors (e.g., ipilimumab, tremelimumab, etc.); and LAG-3 inhibitors (e.g., relatlimab, opdualag, etc.); and combinations thereof. In a specific embodiment, the checkpoint inhibitor is a PD-L1 inhibitor.

In another embodiment, a method of sensitizing a tumor to radiation in a subject in need thereof is provided, the method comprising administering to the subject an effective amount of a compound according to any of the embodiments of Formula I disclosed herein, or a pharmaceutically acceptable salt, racemate, or enantiomer thereof.

In another embodiment, a method of sensitizing a tumor to immunotherapy or chemotherapy in a subject in need thereof is provided, the method comprising administering to the subject an effective amount of a compound according to any of the embodiments of Formula I disclosed herein, or a pharmaceutically acceptable salt, racemate, or enantiomer thereof.

It is well known in the field that benzodiazepine drugs have utility in treating various neurological conditions. Thus, in another embodiment, a method of treating a neurological condition associated with Type-A GABA neurotransmitter receptor function in a subject in need thereof is provided, the method comprising administering to the subject an effective amount of a compound according to any of the embodiments of Formula I disclosed herein, or a pharmaceutically acceptable salt, racemate, or enantiomer thereof.

In a specific embodiment, the neurological condition is selected from the group consisting of sleep disorder, generalized anxiety disorder, social anxiety disorder, seizure disorder, panic disorder, tic disorder, bipolar disorder, and alcohol withdrawal. In a more specific embodiment, the sleep disorder is insomnia. In another more specific embodiment, the seizure disorder is epilepsy.

### EXAMPLES

The following examples are given by way of illustration are not intended to limit the scope of the disclosure.

### Example 1. Materials and Methods

### Cell Lines and Culture Conditions

Cell lines tested were purchased from the American Type Culture Collection (ATCC). Cells were maintained in Dulbecco's Modified Eagle's Medium (DMEM) (Corning) or Roswell Park Memorial Institute (RPMI) 1640 Medium (Gibco). Media for lines was supplemented with 10% (v/v) Fetal Bovine Serum (FBS) (Corning) and 100 U/mL penicillin/streptomycin (Sigma). Lines were grown at 37° C. with 5% (v/v) CO₂. Cells were passaged twice a week at 1:10 ratio and reached 80 to 90% confluency between 72 and 96 hours.

### Drug Preparation

For in vitro studies drug was solubilized in dimethyl sulfoxide (DMSO, 0.125%) prior to use. For mouse studies drug was solubilized in a co-solvent formulation: propylene glycol (40%); ethanol (10%); benzyl alcohol (2%); benzoic acid (2%); sodium benzoate (2%).

### Electrophysiology

Functional characterization employed a Port-a-Patch single cell automated patch-clamp electrophysiology instrument (Nanion Technologies, Germany). Recording solutions were purchased from Nanion Technologies. The composition of external solution consisted of: 140 mM NaCl; 4 mM KCl; 1 mM MgCl₂; 2 mM CaCl₂; 10 mM HEPES; 5 mM D-Glucose. High Ca²⁺ seal enhancer solution was composed of: 130 mM NaCl; 4 mM KCl; 1 mM MgCl₂; 10 mM CaCl₂); 10 mM HEPES; 5 mM D-Glucose. The internal solution composition was: 110 mM KF; 10 mM NaCl; 10 mM KCl; 10 mM EGTA; 10 mM HEPES, pH 7.2 adjusted using KOH.

To increase the current amplitude in single cell recordings, GABA and QH-II-066 were dissolved in high sodium containing external solution composed of: 161 mM NaCl; 3 mM KCl; 1 mM MgCl₂; 1.5 mM CaCl₂); 10 mM HEPES; 6 mM D-Glucose. Whole-cell recordings were performed on cells (held at −80 mV) using a gap-free protocol under continuous perfusion of external solutions and drug applications. GABA (1 μM) and QH-II-066 (4 μM) were applied briefly for 5 sec to record the current potentiation. Data acquisition was obtained using HEKA Elektronik software (Dr. Schulze GmBH, Germany). Data were low-pass filtered at 1 kHz and digitalized at 100 kHz. Data analysis was performed by computing the maximum current amplitude using Nest-O-Patch Software (Open Source).

### Mitochondrial Depolarization

Drug was diluted to 4 μM in RPMI-1640 culture media. Carbonyl cyanide 4-(trifluoromethoxy) phenylhydrazone (FCCP) (Sigma) was diluted to 20 μM in culture medium. Tetramethylrhodamine ethyl ester perchlorate (TMRE) (Sigma) was diluted to 400 nM in culture medium. H1792 cells were grown in culture to 75-90% confluency. Cells (5×105 cells/mL) were harvested and resuspended in media. Cell suspension (200 μL) was dispensed and drug or FCCP (200 μL) added to final concentrations of 2 μM and 10 μM, respectively. Solution was briefly vortexed and incubated for 10 min. TMRE (40 μL of 400 nM stock) was added (final concentration 10 nM), vortexed, and sample reading acquired using a BD LSR Fortessa (Beckton Dickinson, San Diego). Data was analyzed using Flowjo v10 software (Flowjo, LLC).

### Immunoblotting

DNA was sheared by sonication. Lysates were kept on ice for 30 min, centrifuged 10 min (13,500×g, 4° C.), and protein concentration of supernatant determined by a Bradford assay (Protein Assay Dye Reagent, Bio-Rad). Lysates were mixed 1:1 with 2× Laemmli sample buffer containing β-mercaptoethanol and heated 5 min at 95° C. Protein was resolved by SDS-PAGE using 4-20% gradient polyacrylamide gels (Bio-Rad), then transferred to nitrocellulose membranes (Bio-Rad) for 2 hr at 100 V in tris-glycine transfer buffer containing 20% methanol. Membranes were blocked at room-temperature in 5% 1×TBST blocking buffer (TBS with 0.1% Tween-20 and 5% non-fat dry milk) for 1 hr with gentle agitation, followed by overnight incubation with primary Ab and gentle shaking at 4° C. The primary Abs were diluted as follows: GAPDH (1:1000, Cell Signaling Technology); GABARAP (1:1000, Cell Signaling Technology); NIX (1:1000, Cell Signaling Technology). Immunodetection was performed with anti-rabbit horseradish-peroxidase-conjugated secondary antibody (1:10000, Cell Signaling Technology). Post-primary Ab incubation, membranes were washed (3× for 10 min in TBST at room-temperature), probed with rabbit HRP tagged secondary antibody (1:3000, Cell Signaling Technology), and processed for chemiluminescence detection using ECL kit (Thermo Scientific). Chemiluminescence images were acquired using ChemiDoc Touch Imaging System (Bio-Rad).

### Immunofluorescence Microscopy

Cells were seeded on sterile glass cover slips and grown overnight at 37° C. Cells were then rinsed with cold PBS and fixed in 4% paraformaldehyde (30 min, room temperature). Fixed cells were rinsed in PBS (3×5 min at room-temperature), and permeabilized (15 min in PBS containing 0.1% Triton X-100). Cells were washed in PBS (3× for 5 min at room-temperature) and incubated with blocking buffer (PBS containing 0.3% Triton X-100 and 3% BSA) for 1 hr at room-temperature with gentle shaking. Blocking solution was aspirated, cells washed with ice-cold PBS, and incubated overnight with primary Ab (LC3B or NIX) diluted 1:200 in sterile 0.5% BSA in PBS at 4° C. on the cover slip placed on a glass slide kept inside a humidified 10 cm dish with gentle shaking 60. Cells were washed (3× for 5 min in PBS) and incubated with fluorophore conjugated secondary Ab goat anti-rabbit Alexa Fluor 594 (Abcam) in 2% normal donkey serum at room-temperature for 1 hr in dark. Cells were washed (3× for 5 min in PBS) under low light and mounted on a glass slide in VECTASHIELD Antifade Mounting Medium with DAPI (Vector Laboratories). The following Abs were used (1:200 dilution): rabbit anti-human LC3B (Cell Signaling Technology) and rabbit NIX (Cell Signaling Technology). Slides were imaged on a Zeiss LSM 710 laser scanning confocal microscope and analyzed using NIH Fiji ImageJ2 (version 1.53c).

### In Vitro Viability Assays

For cell proliferation viability studies, cells were assayed using the Cell Titer 96® Aqueous One Solution Assay (Promega). IC₅₀ values were determined using the ‘[Inhibitor] versus normalized response’ nonlinear regression function and the log of inhibitor concentration in Prism 8 software (version 8.3.0 for MacOS).

In Vitro Cell Survival Assay with Inhibitory Peptide

H1792 cells (2500 cells per well in 100 μL media) were plated in 96 well plates with colorless phenol-red free RPMI-1640 media and allowed to grow overnight. On the next day, the media was removed and inhibitory peptide pen-3-ortho (gift of J. Kritzer, Tufts University) diluted in fresh 100 μL of phenol-red free RPMI media was added in two sets of wells (6 wells in each set) at two different concentrations, 15 μM and 25 μM.

Cells in another set of 6 wells were treated with 3 μM QH-II-066 in 100 μL phenol-red free media in each well. Two other treatment groups (each group having 6 wells) were treated with a combination of QH-II-066 (3 μM) and pen-3-ortho inhibitory peptide in two different concentrations, 15 μM and 25 μM. One set of 6 wells with cells were kept as control with no treatment and one set of 6 cells was kept as media only control with no cells plated in them. After adding the drug and DMSO (in control) all groups of cells including the controls, treated and media-only groups in the culture plate were incubated at 37° C., 5% CO₂ in a humidified environment in an incubator for 48 hours. Following the 48-hr incubation, 20 μL of diluted MTS reagent was added to each control (DMSO) and treated group of wells and in the wells containing only media. The cells were incubated at 37° C. for 1 hr and the absorbance were measured at 490 nm using a microplate reader (Molecular Devices).

The mean of the absorbance of media only samples was calculated, and the value was subtracted from each control and test samples. The percentage of inhibition in each group was calculated by the formula (C−T)/C×100% where “C” is the mean absorbance reading for Control group, “T” is the mean absorbance reading for each treated group. The percentage of survival was calculated by subtracting percentage of inhibition of each group from 100%. Percentage survival data is expressed as mean±SEM, analyzed with GraphPad Prism 8.0.1 software (San Diego, CA, USA). Student's t test (paired) for two groups were used for statistical comparison. p<0.05 was significant.

### Mouse Experiments

For subcutaneous xenograft tumor growth delay experiments, black 6 mice (C57Bl6) were purchased (Charles River Laboratories) and housed at University of Cincinnati LAMS. Purchased mice were allowed to accommodate for a week prior to experiment. Mice were housed in pathogen-free rooms and clinical health evaluated weekly by veterinary staff of the University of Cincinnati LAMS. All animal studies were conducted in accordance with approval of a University of Cincinnati IACUC.

LLC1 cells (a million) grown in RPMI medium were washed in cold PBS and mixed with Matrigel 25%, then injected subcutaneously into left and right flanks above the hind limbs of 6 to 8-week-old mice. When subcutaneous tumors were palpable (˜100 mm³ in size) the following treatments groups were initiated: (1) vehicle (2) drug. In mice receiving vehicle alone, the vehicle was injected i.p for 7 days. In mice receiving drug, drug (2.5 mg/kg body weight) was injected i.p. for 7 days. Following end of treatment, tumor volume were taken for growth delay studies. Mouse tumors were measured by Vernier calipers. Tumor volume was calculated using the formula: 4/3π×(l×h2), where: l and h are radii of the tumor taken perpendicular to each other. Tumor size was measured three times a week using a caliper.

### Example 2. GABA_{A} Receptors are Functional in Cancer Cells

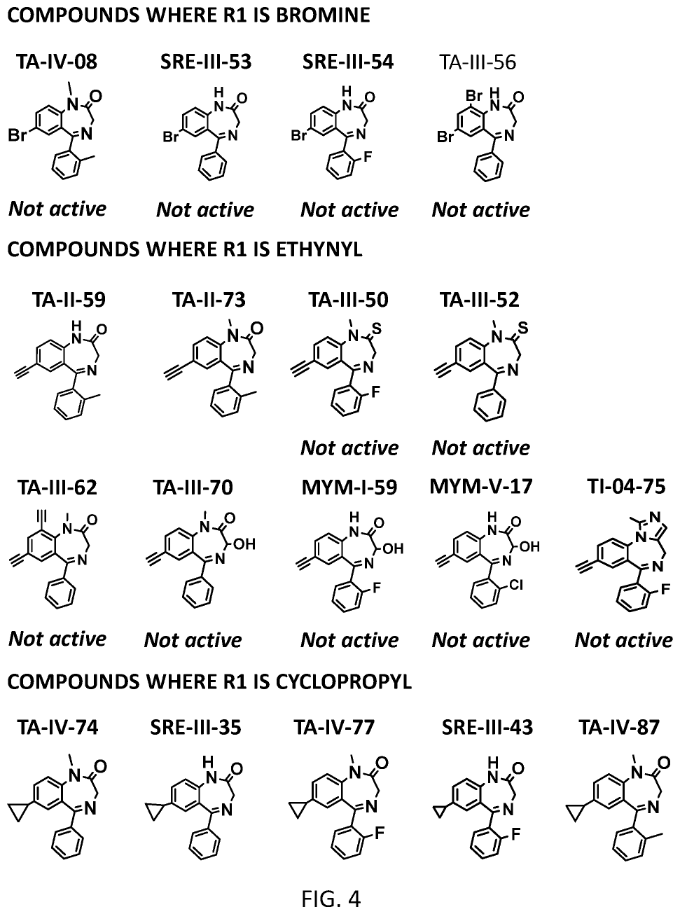

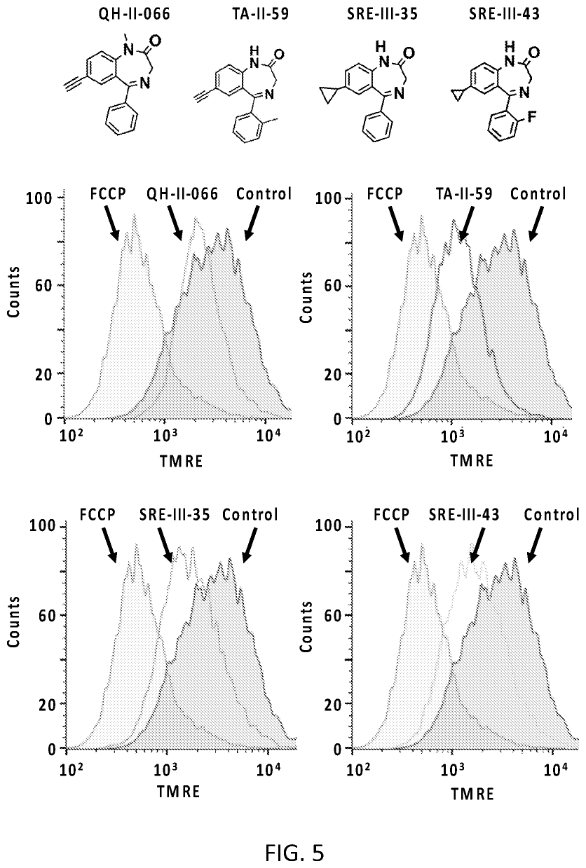

Patch clamp electrophysiology of single primary patient-derived cancer cells from lung adenocarcinoma was employed to demonstrate intrinsic, functional GABA_{A} receptors. A current signal was observed in response to GABA, as illustrated for lung adenocarcinoma cell line H1792 (FIG. 4). A single molecule of benzodiazepine binds per canonical site on GABA_{A} receptors and functions to enhance the effect of GABA. An enhanced response is observed to GABA plus QH-II-066 (1 and 4 μM, respectively) over GABA (1 μM) alone in the lung cancer cells (FIG. 3). Similarly, medulloblastoma (cells and tumor tissue) and melanoma cells have intrinsic, functional GABA_{A} receptors and QH-II-066 enhances chloride transport. This functional analysis reveals that these cancer cells possess: (1) intrinsic functional GABA_{A} receptors; and (2) receptors that form a canonical assembly with an α-γ interface, given that a benzodiazepine can bind and elicit a positive response.

### Example 3. Enhanced GABA_{A} Receptor Activity is Depolarizing to Cancer Cells

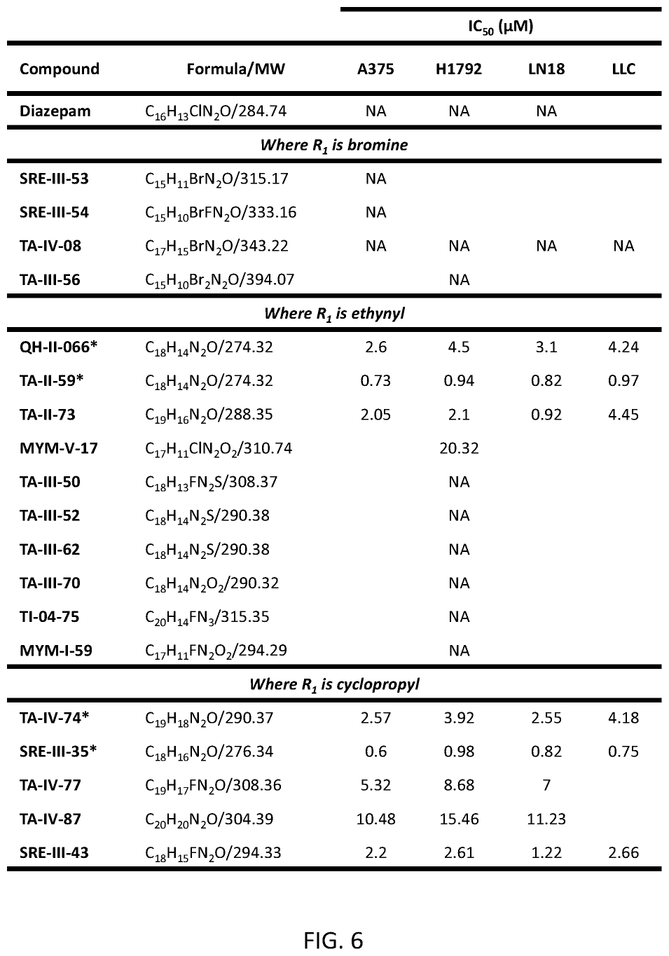

QH-II-066 mediates enhanced GABA_{A} receptor activation and leads to an efflux of chloride anions across the extracellular plasma membrane, which contributes to a depolarization of the mitochondrial transmembrane. QH-II-066 creates a shift in electric charge distribution in different cancer cell types. Newly synthesized benzodiazepine analogs TA-II-59; SRE-III-35; SRE-III-43 (FIG. 4) also create a shift in electric charge distribution that is depolarizing to disparate cancer cells (See FIG. 5).

### Example 4. Enhanced GABA_{A} Receptor Activity Triggers Cell Death

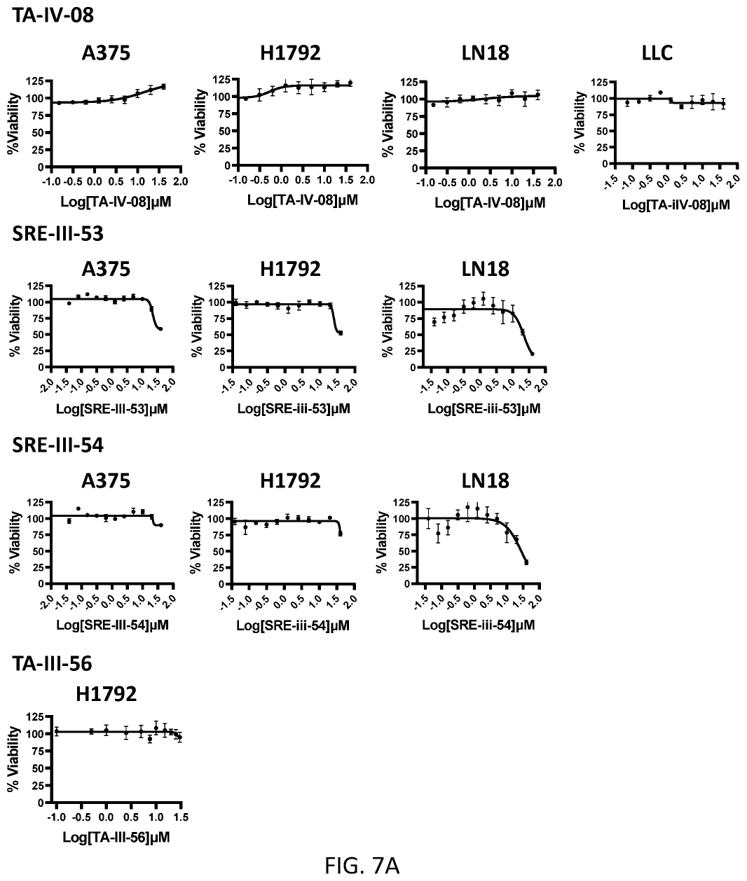

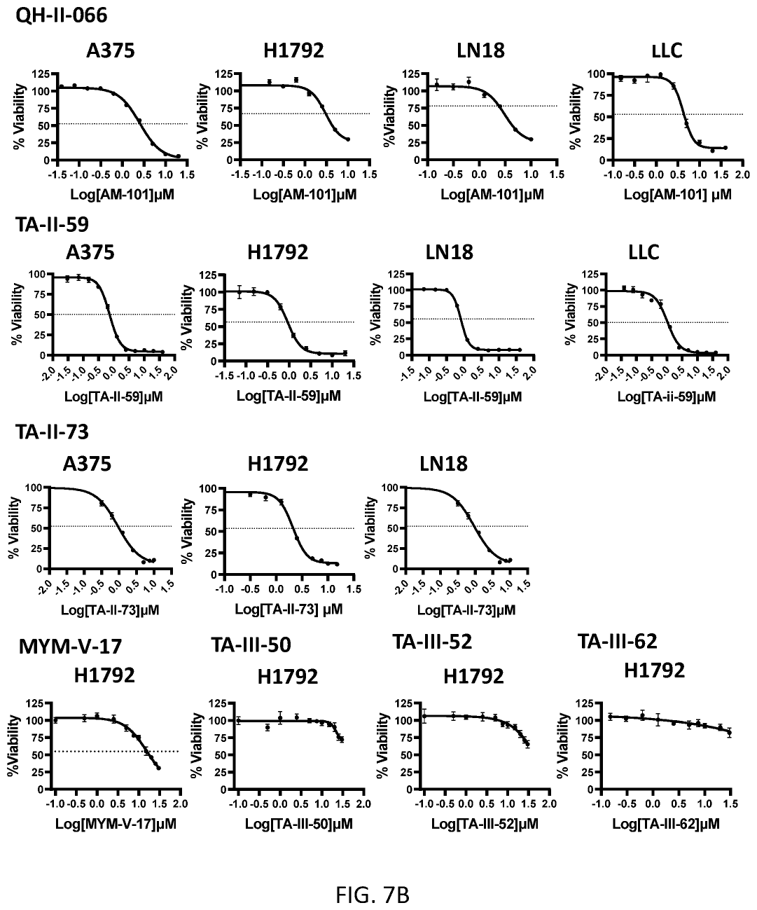

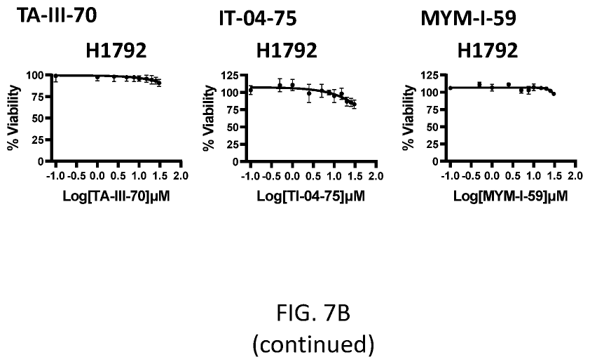

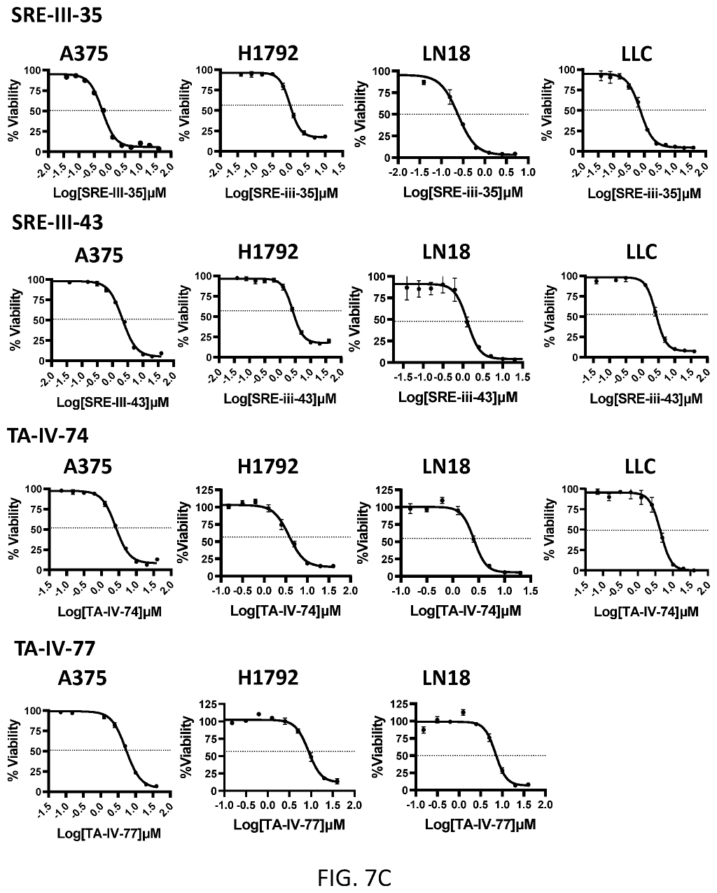

Depolarization can trigger cell death via activation of the intrinsic (mitochondrial) apoptotic pathway and we have reported this phenomenon in cell lines of the pediatric brain cancer medulloblastoma and melanoma using QH-II-066. We report here an examination of eighteen novel benzodiazepine analogs for their ability to impair the viability of cancer cells. These benzodiazepine analogs can be grouped into three classes based on the R1 moiety: (1) bromine; (2) ethynyl; (3) cyclopropyl (FIG. 4). Majority of these compounds were tested in four cell lines: three patient-derived cell lines representing glioma (LN18), lung adenocarcinoma (H1792), melanoma (A375); one lung cancer mouse line, Lewis Lung Carcinoma or LLC1. None of the compounds having an R1 bromine are cytotoxic to cancer cells (FIG. 6; FIG. 7A), which reflects that the identity at this position is critical to elicit a cytotoxic response in cancer cells. In contrast, compounds with either the ethinyl or cyclopropyl are cytotoxic (FIG. 6; FIGS. 7B, C). However, changing of other chemical groups in the benzodiazepine can negate this effect. For example, sulfur (TA-III-50 and TA-III-52), bromine (TA-III-56), a second ethynyl (TA-III-62), OH (TA-III-70 and MYM-I-59) are poorly or inactive (FIG. 4; FIG. 6; FIG. 7B, C). Amongst the novel ethynyl compounds tested, TA-II-59 was the most potent and consistently so in different cell lines. Furthermore, TA-II-59 was an order of magnitude more potent than QH-II-066. The cyclopropyl compounds reported here were also cytotoxic in the different cancer cell lines tested (FIG. 6; FIG. 7C). SRE-III-35 was equally as potent as TA-II-59. Both SRE-III-35 and TA-II-59 are NOR-variants of TA-IV-74 and TA-II-73, respectively. And both these NOR-variants are 2-fold more cytotoxic than the non-NOR-variants.

### Example 5. New Benzodiazepine Analog are Potent In Vivo

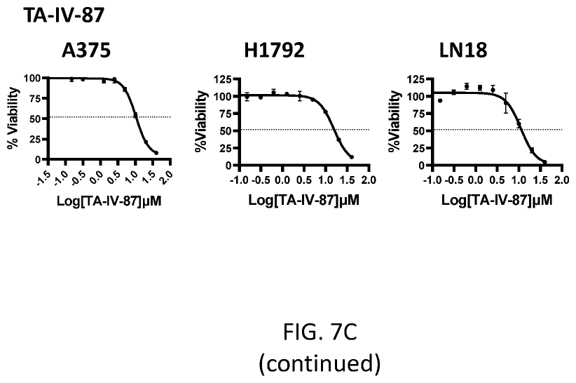

Mouse efficacy studies of three of the novel benzodiazepine variants were conducted: the ethynyl variant TA-II-59; and cyclopropyl variants SRE-III-35 and TA-IV-74. Xenograft tumors were generated in left and right flanks of black 6 mice using LLC1 cells. The treatment protocol involved administering a single i.p. dose of drug (2.5 mg drug/kg body weight) for seven consecutive days, once the tumor was palpable (FIG. 8). All of the three new variants as well as QH-II-066 result in significant tumor control relative to the control or vehicle mice (FIG. 8). In contrast to what was observed in vitro, the NOR-variants do not show the most significant effect in vivo. While not desiring to be bound by theory, this may reflect an improved bioavailability of the non-NOR-variants, which enhances their effectiveness. The most significant effect was observed in mice administered TA-IV-74. These mice showed no increase in tumor volume at 25 days.

### Example 6. GABA_{A} Receptor Activation Enhances Autophagy

The contribution of molecular events to the observed cytotoxic effect of this new class of benzodiazepine analogs has been examined, i.e., those containing an ethynyl. In considering how this class of benzodiazepines may mediate cell death and tumor control, several observations suggested that autophagy may be a contributing factor. First, these compounds depolarize the mitochondrial transmembrane. It has been reported that depolarization of mitochondria serves to trigger autophagy. Second, a central protein to autophagy induction is GABARAP, which prior to its role in autophagy was shown to interact with GABA_{A} receptors. GABARAP is also a NIX interacting factor, which has a significant role in autophagosome formation.

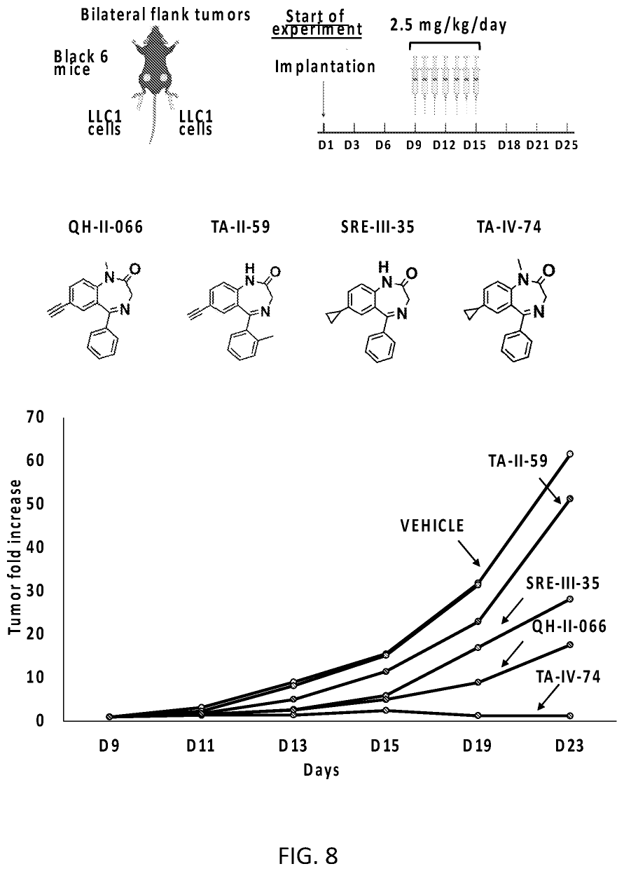

Key proteins to the assembly of autophagy granules or puncta, a hallmark of autophagy induction, are the ATG8 sub-family associated protein LC3B and NIX. Importantly, NIX mediates an association between the mitochondria and GABARAP, which is associated with the GABA_{A} receptor at the extracellular plasma membrane. Subcellular distribution of autophagosomal proteins by immunofluorescence (IF) (e.g., LC3 puncta formation) is a well-established assay to monitor autophagy. Confocal IF was employed to observe assembly of puncta in lung adenocarcinoma cells (H1792) treated with QH-II-066. There is a background level of both LC3B- and NIX-positive puncta in H1792 cells in the control (DMSO treated) group (FIG. 9). However, quantification of puncta reveals that QH-II-066 alone creates an environment for increased puncta positive for both markers.

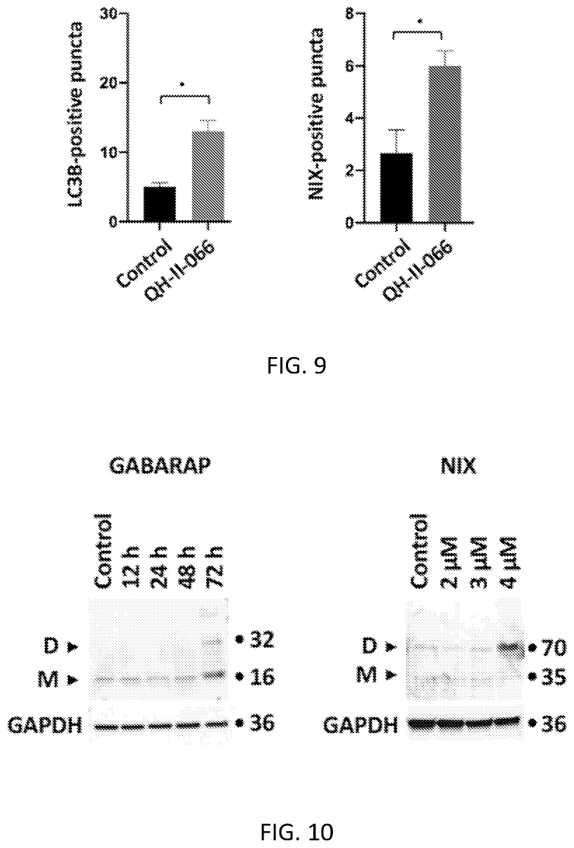

Having observed enhanced assembly of LC3B and NIX-positive puncta, assays focused on the protein GABARAP, since NIX binds to GABARAP. In a time-course experiment it was found that lung adenocarcinoma cells (H1792) treated with QH-II-066 show a pronounced accumulation of both GABARAP and NIX at 72 hours post-treatment. Importantly, GABARAP and NIX also dimerize at 72 hours (FIG. 10). This is the time point when lung adenocarcinoma cell viability is impaired by QH-II-066. Furthermore, QH-II-066 induced NIX dimerization is dose-dependent (FIG. 10).

### Example 7. GABARAP Structural Perturbation Inhibits GABA_{A} Receptor Mediated Cytotoxicity Commensurate with NIX Destabilization

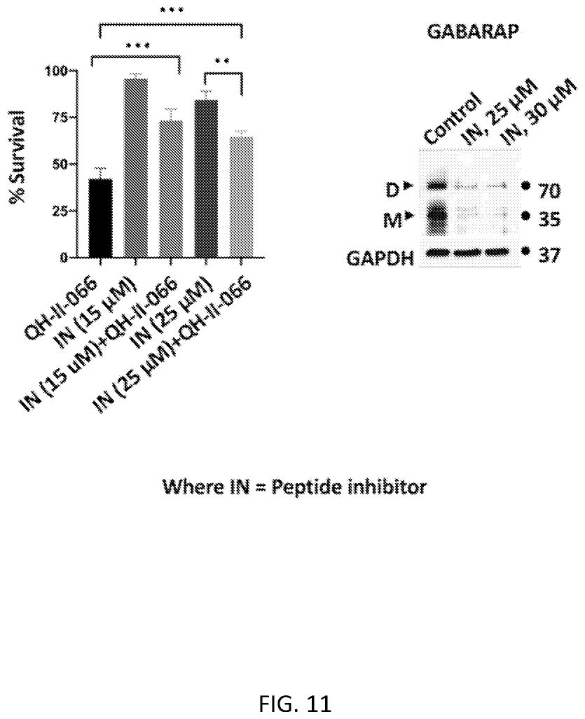

To establish that the autophagic response that was observed contributed to the cytotoxicity of QH-II-066, a peptide designed to target the protein GABARAP was employed and reported to inhibit autophagy in ovarian cancer cells. This peptide, pen-3-ortho (gift of J. Kritzer, Tufts University), has several advantages: (1) it is highly specific for GABARAP, binding with a low nanomolar affinity; and (2) a crystal structure has been determined of pen-3-ortho in complex with GABARAP, thus its mode of action delineated. Mechanistically, pen-3-ortho is a competitive inhibitor of NIX for binding to GABARAP. Pen-3-ortho alone is not cytotoxic to H1792 cells or nominally at exceedingly high concentrations (FIG. 11, left panel). Next, the investigators examined whether pen-3-ortho inhibited the cytotoxic response of QH-II-066. Pen-3-ortho combined with QH-II-066 significantly inhibits cytotoxicity of QH-II-066 on H1792 cells. This inhibitory effect is concentration-dependent. Western blotting of cells treated with pen-3-ortho shows significantly less NIX protein, both monomer and dimer states (FIG. 11, right panel). This supports published work that GABARAP interacts with and stabilizes NIX. Importantly, these experiments support GABARAP as a mediator between GABA_{A} receptor function and autophagosome assembly.

### Example 8. Model of Mechanism of Action

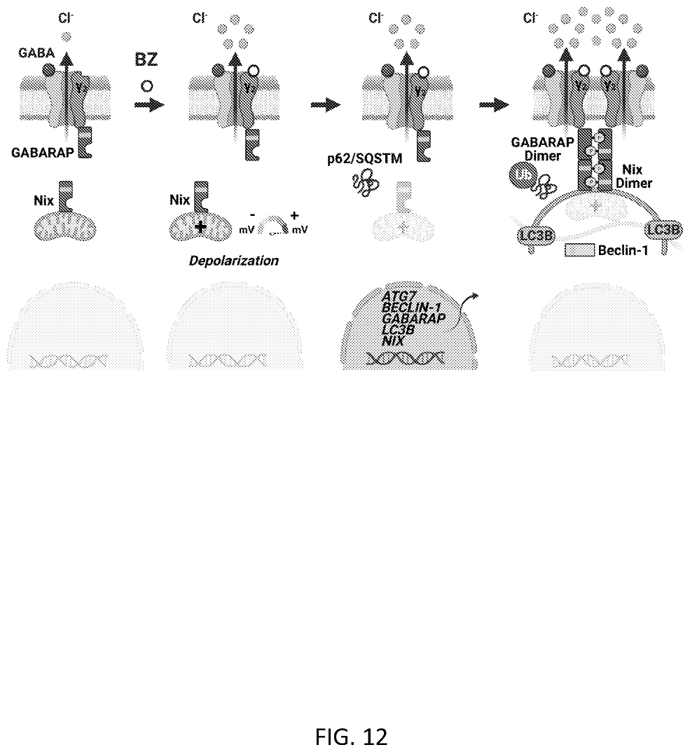

It has previously been reported that, in cancer cells, GABA_{A} receptors are functional and that their activation using a member of our benzodiazepine analogs enhances the effect of its natural agonist, GABA (FIG. 12). Previously, it was found that activation of GABA_{A} receptors in cancer cells contributes to an efflux of chloride anions. It was also found that GABA_{A} receptor activation leads to depolarization of the mitochondrial transmembrane, consistent with a net efflux of chloride anions. Depolarization has been reported to lead to induction of autophagy. GABARAP and NIX are proteins key to the nucleation of autophagosome assembly and bridging the extracellular plasma membrane and the mitochondrial transmembrane. Specifically, the GABARAP subfamily of proteins promote autophagy by regulating the activity of kinase ULK1, a key mediator of autophagy whose function stabilizes autophagosome formation. In addition, phosphorylated GABARAP traffics GABA_{A} receptors to the extracellular plasma membrane, binding to its 72-subunit. While NIX is an autophagy receptor that rests in the mitochondrial transmembrane, and complexes with GABARAP to recruit mitochondria to autophagosomes.

As well as enhancing expression of GABRARAP and Nix, the investigators found that GABA_{A} receptor activation contributes to their multimerization, thus augmenting the autophagic response. While not desiring to be bound by theory, the data suggests a model wherein multimerization of GABARAP occurs commensurate with multimerization of GABA_{A} receptors, and that this macromolecular assembly assumes a cytotoxic-state as it drives a significant efflux of chloride anion locally. This model is consistent with experimental analysis and theoretical modeling, which shows that multimerization of GABA_{A} receptors, and in turn GABARAP, generates an enhanced localized charge differential. This would also occur commensurate with a stabilization and multimerization of NIX, as observed. In this way, perturbation of ion homeostasis sensed and regulated by GABA_{A} receptors would lead to assembly of an autophagosome and potential enclosure and subsequent recycling of mitochondria.

Aspects of the present disclosure can be described with reference to the following numbered clauses, with preferred features laid out in dependent clauses.

1. A compound according to Formula I, or a pharmaceutically acceptable salt, racemate, or enantiomer thereof:

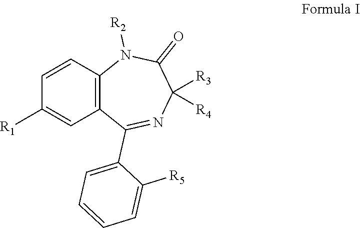

wherein: R₁ is selected from the group consisting of C₂-C₄ alkynyl, C₃-C₆ cycloalkyl, d(5)-cyclopropyl, methyl alkynyl, alkynyl-CD₃, and alkynyl-CF₃; R₂ is selected from the group consisting of hydrogen, methyl, and trideuteromethyl; R₃ and R₄ are independently selected from the group consisting of hydrogen, deuterium, and methyl; and R₅ is selected from the group consisting of hydrogen, methyl, trideuteromethyl, tritritiomethyl, halogen, and trifluoromethyl; optionally, wherein when R₂ is trideuteromethyl, R₁ is not ethynyl; and optionally, wherein when R₁ is ethynyl, R₅ is not hydrogen.

2. The compound according to clause 1, wherein R₃ and R₄ are each hydrogen.

3. The compound according to clause 1, wherein R₃ and R₄ are each deuterium.

4. The compound according to clause 1, wherein R₃ is hydrogen and R₄ is methyl.

5. The compound according to any of the preceding clauses, wherein R₅ is Cl, F, or Br.

6. The compound according to any of the preceding clauses, wherein R₁ is cyclopropyl.

7. The compound according to clause 6, wherein the compound is selected from the group consisting of:

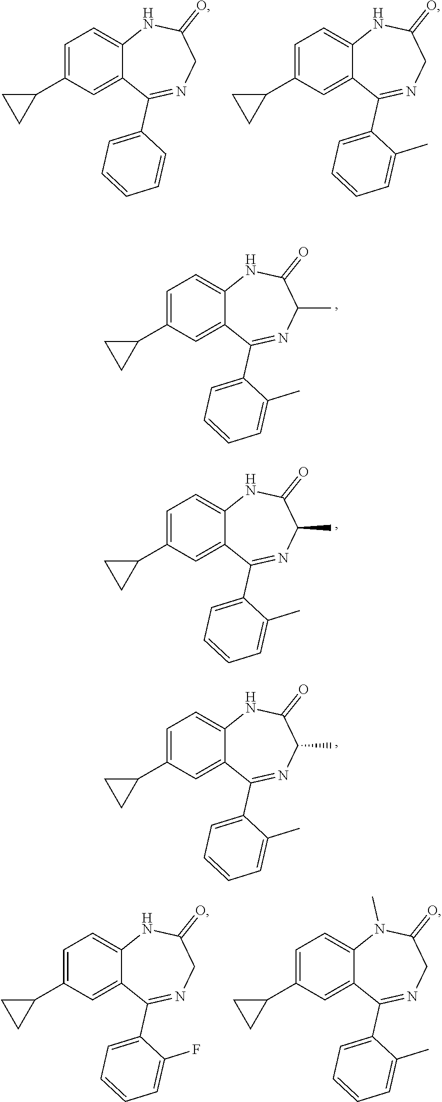

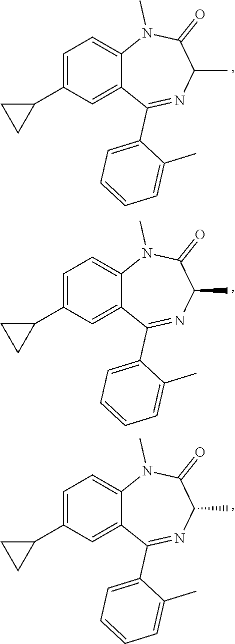

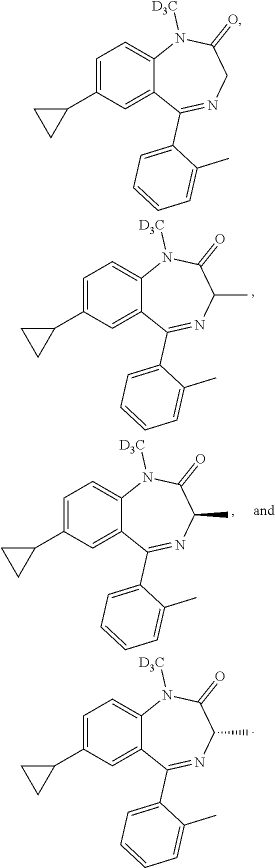

8. The compound according to any of clauses 1-7, wherein the compound is selected from Table 1.

9. The compound according to any of clauses 1-7, wherein the compound is selected from the group consisting of:

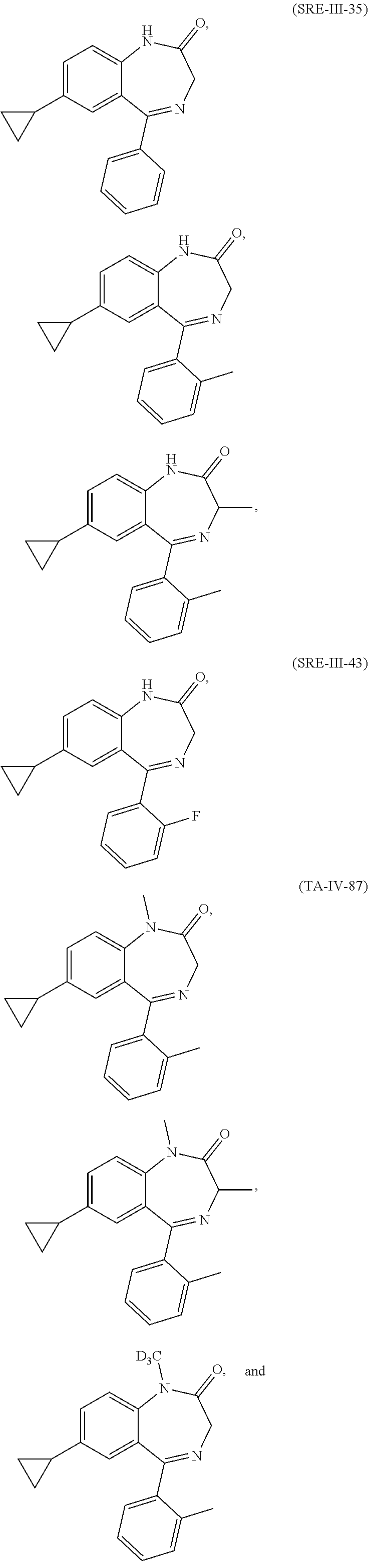

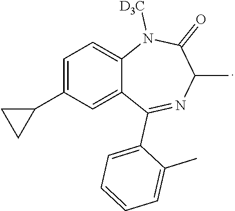 [SMILES: [2H]C([2H])([2H])N1C(=O)C(C)N=C(c2ccccc2C)c2cc(C3CC3)ccc21]

10. The compound according to any of clauses 1-5, wherein R₁ is ethynyl.

11. The compound according to clause 10, wherein the compound is selected from the group consisting of:

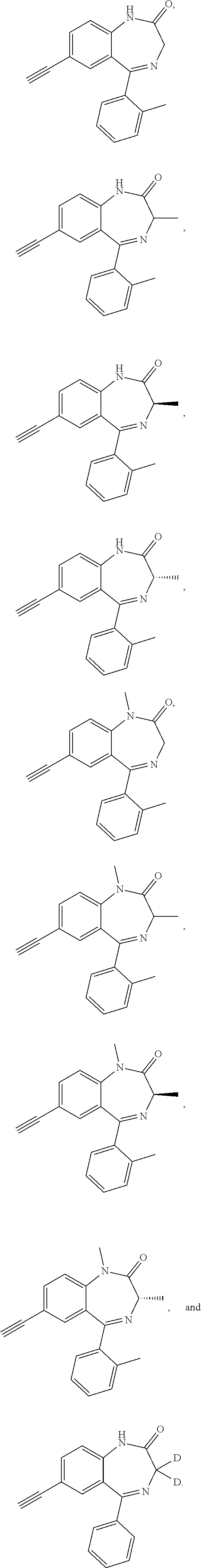

12. The compound according to clause 10, wherein the compound is selected from the group consisting of:

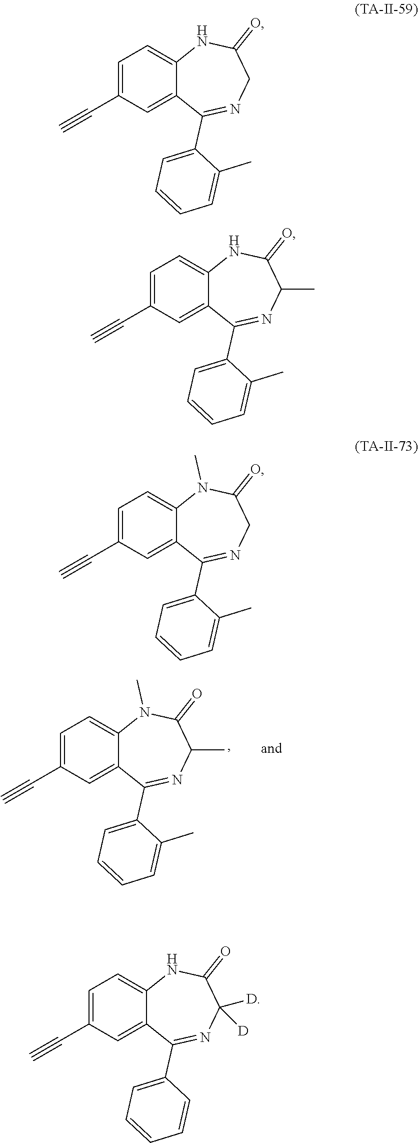

13. The compound according to any of the preceding clauses, wherein R₁ is cyclopropyl or ethynyl; R₂ is hydrogen or methyl; R₃ and R₄ are each deuterium; and R₅ is selected from the group consisting of hydrogen, methyl, trideuteromethyl, tritritiomethyl, trifluoromethyl, fluorine, and chlorine.

14. A pharmaceutical composition comprising:
an effective amount of the compound according to any of clauses 1-13; and
a pharmaceutically acceptable carrier.

15. A method of treating cancer in a subject in need thereof, the method comprising administering to the subject an effective amount of a compound according to any of clauses 1-13 or the pharmaceutical composition of clause 14.

16. A method of sensitizing a tumor to radiation in a subject in need thereof, the method comprising administering to the subject an effective amount of the compound according to any of clauses 1-13 or the pharmaceutical composition of clause 14.

17. A method of sensitizing a tumor to immunotherapy or chemotherapy in a subject in need thereof, the method comprising administering to the subject an effective amount of the compound according to any of clauses 1-13 or the pharmaceutical composition of clause 14.

18. A method of treating a neurological condition associated with Type-A GABA neurotransmitter receptor function in a subject in need thereof, the method comprising administering to the subject an effective amount of the compound according to any of clauses 1-13 or the pharmaceutical composition of clause 14.

19. The method according to clause 18, wherein the neurological condition is selected from the group consisting of sleep disorder, generalized anxiety disorder, social anxiety disorder, seizure disorder, panic disorder, tic disorder, bipolar disorder, and alcohol withdrawal.

20. The method according to clause 19, wherein the sleep disorder is insomnia.

21. The method according to clause 19, wherein the seizure disorder is epilepsy.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

The foregoing description is illustrative of particular embodiments of the invention but is not meant to be a limitation upon the practice thereof. While particular embodiments have been illustrated and described, it would be obvious to one skilled in the art that various other changes and modifications can be made without departing from the spirit and scope of the invention. It is therefore intended to cover in the appended claims all such changes and modifications that are within the scope of this invention.

1. A compound according to Formula I, or a pharmaceutically acceptable salt, racemate, or enantiomer thereof: 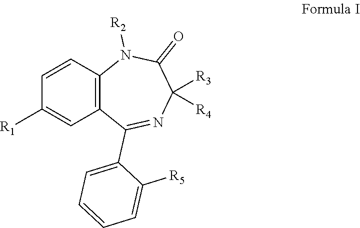
wherein:
R₁ is selected from the group consisting of C₂-C₄ alkynyl, C₃-C₆ cycloalkyl, d(5)-cyclopropyl, methyl alkynyl, alkynyl-CD₃, and alkynyl-CF₃;
R₂ is selected from the group consisting of hydrogen, methyl, and trideuteromethyl;
R₃ and R₄ are independently selected from the group consisting of hydrogen, deuterium, and methyl; and
R₅ is selected from the group consisting of hydrogen, methyl, trideuteromethyl, tritritiomethyl, halogen, and trifluoromethyl;
wherein when R₂ is trideuteromethyl, R₁ is not ethynyl; and
wherein when R₁ is ethynyl, R₅ is not hydrogen.

2. The compound according to claim 1, wherein R₃ and R₄ are each hydrogen or are each deuterium.

3. (canceled)

4. The compound according to claim 1, wherein R₃ is hydrogen and R₄ is methyl.

5. The compound according to claim 1, wherein R₅ is Cl, F, or Br.

6. The compound according to claim 1, wherein R₁ is cyclopropyl.

7. The compound according to claim 6, wherein the compound is selected from the group consisting of:

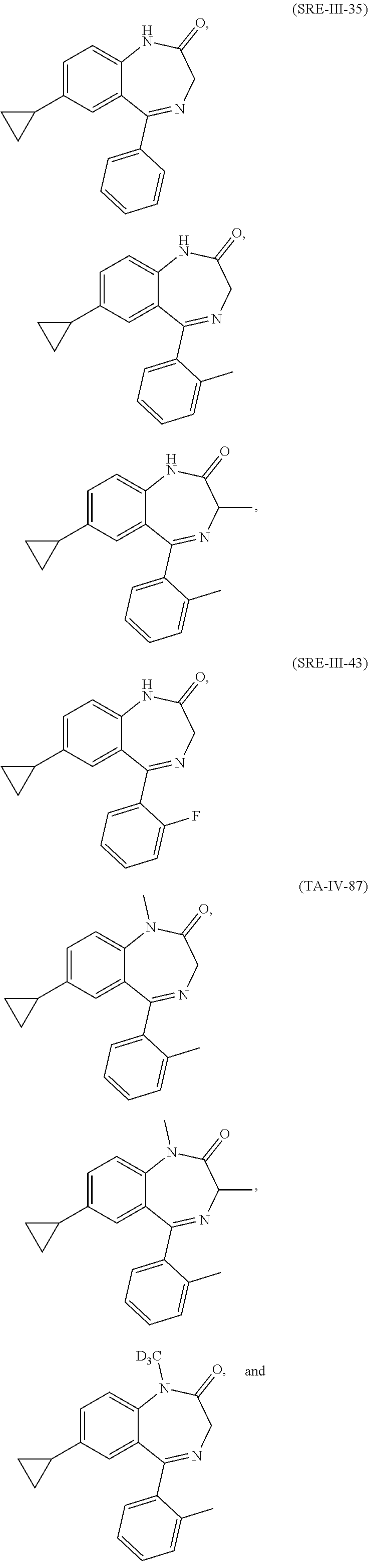

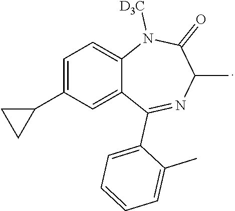 [SMILES: [2H]C([2H])([2H])N1C(=O)C(C)N=C(c2ccccc2C)c2cc(C3CC3)ccc21]

8. The compound according to claim 1, wherein R₁ is ethynyl.

9. The compound according to claim 8, wherein the compound is selected from the group consisting of:

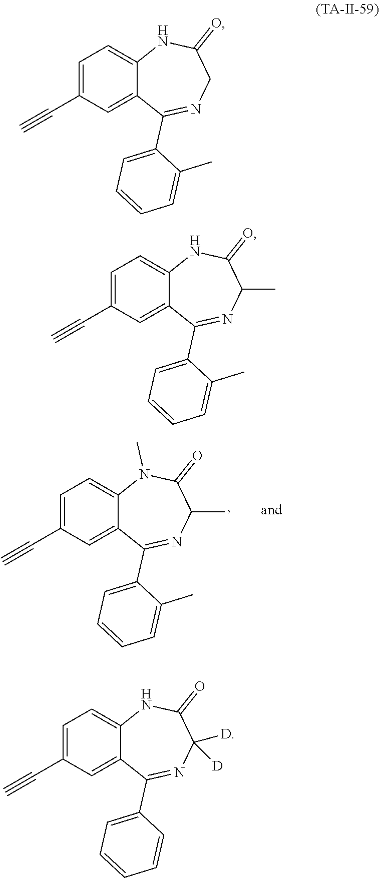

10. The compound according to claim 1, wherein R₁ is cyclopropyl or ethynyl; R₂ is hydrogen or methyl; R₃ and R₄ are each deuterium; and R₅ is selected from the group consisting of hydrogen, methyl, trideuteromethyl, tritritiomethyl, trifluoromethyl, fluorine, and chlorine.

11. A pharmaceutical composition comprising:
an effective amount of the compound according to claim 1; and
a pharmaceutically acceptable carrier.

12. A method of treating cancer in a subject in need thereof, the method comprising administering to the subject an effective amount of a compound according to Formula I, or a pharmaceutically acceptable salt, racemate, or enantiomer thereof:
wherein:
R₁ is selected from the group consisting of C₂-C₄ alkynyl, C₃-C₆ cycloalkyl, d(5)-cyclopropyl, methyl alkynyl, alkynyl-CD₃, and alkynyl-CF₃;
R₂ is selected from the group consisting of hydrogen, methyl, and trideuteromethyl;
R₃ and R₄ are independently selected from the group consisting of hydrogen, deuterium, and methyl; and
R₅ is selected from the group consisting of hydrogen, methyl, trideuteromethyl, tritritiomethyl, halogen, and trifluoromethyl;
wherein when R₂ is trideuteromethyl, R₁ is cyclopropyl; and
wherein when R₁ is ethynyl, R₅ is not hydrogen.

13. The method according to claim 12, wherein R₃ and R₄ are each hydrogen or are each deuterium.

14. (canceled)

15. The method according to claim 12, wherein R₃ is hydrogen and R₄ is methyl.

16. The method according to claim 12, wherein R₅ is Cl, F, or Br.

17. The method according to claim 12, wherein R₁ is cyclopropyl.

18. The method according to claim 17, wherein the compound is selected from the group consisting of:

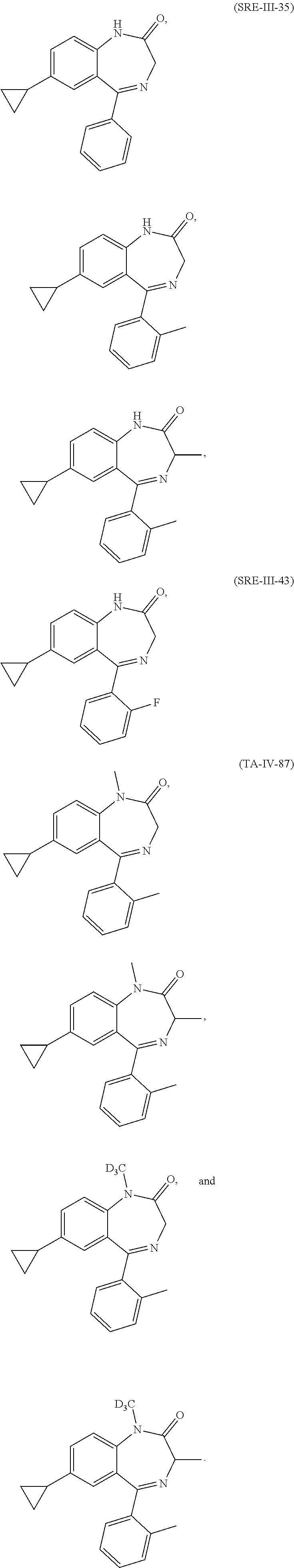

19. The method according to claim 12, wherein R₁ is ethynyl.

20. The method according to claim 19, wherein the compound is selected from the group consisting of:

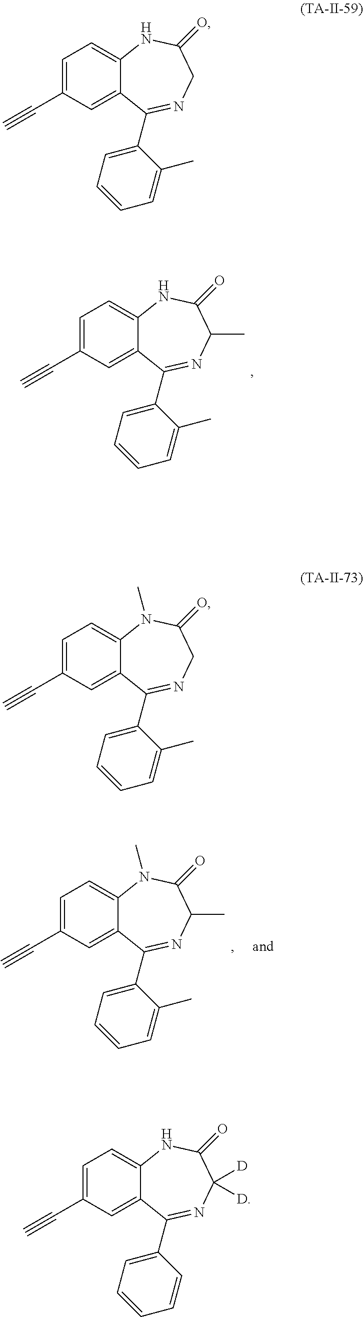

21. A method of sensitizing a tumor to radiation in a subject in need thereof, the method comprising administering to the subject an effective amount of the compound according to claim 1.

22. A method of sensitizing a tumor to immunotherapy or chemotherapy in a subject in need thereof, the method comprising administering to the subject an effective amount of the compound according to claim 1.

23. A method of treating a neurological condition associated with Type-A GABA neurotransmitter receptor function in a subject in need thereof, the method comprising administering to the subject an effective amount of the compound according to claim 1.

24. The method according to claim 23, wherein the neurological condition is selected from the group consisting of sleep disorder, generalized anxiety disorder, social anxiety disorder, seizure disorder, panic disorder, tic disorder, bipolar disorder, and alcohol withdrawal.

25. The method according to claim 24, wherein the sleep disorder is insomnia.

26. The method according to claim 24, wherein the seizure disorder is epilepsy.

27. The compound according to claim 8, wherein when R₃ and R₄ are each hydrogen, R₂ is not methyl.
